(19) United States
(12) Patent Application Publication
(10) Pub. No.: US 2025/0215406 A1
(43) Pub. Date: Jul. 3, 2025

(54) RECOMBINANT REVERSE TRANSCRIPTASE WITH IMPROVED PROCESSIVITY

(71) Applicants: FAPON LIFE SCIENCES INC., Boston, MA (US); GUANGDONG FAPON BIOLOGICAL CO., LTD., Guangdong (CN)

(72) Inventors: Ning WU, Boston, MA (US); Shengxi GUAN, Boston, MA (US)

(21) Appl. No.: 18/666,799

(22) Filed: May 16, 2024

Related U.S. Application Data
(60) Provisional application No. 63/615,793, filed on Dec. 29, 2023.

(51) Int. Cl. C12N 9/12; C07K 14/47

(52) U.S. Cl. CPC C12N 9/1276; C07K 14/4705; C12Y 207/07049

(57) ABSTRACT

The present disclosure provides a recombinant reverse transcriptase with improved processivity and the use thereof. In one embodiment, the recombinant reverse transcriptase is a fusion protein comprising a reverse transcriptase and a cold shock protein.

### CROSS-REFERENCE TO RELATED APPLICATIONS

This application claims priority to U.S. provisional application 63/615,793, filed Dec. 29, 2023, the disclosure of which is incorporated herein by reference.

### SEQUENCE LISTING

The sequence listing that is contained in the file named “088723-8001US01-fixed”, which is 119,195 bytes (as measured in Microsoft Windows) and was created on Jul. 9, 2024, is filed herewith by electronic submission and is incorporated by reference herein.

### FIELD OF THE INVENTION

The present disclosure relates generally to the field of molecular biology. In particular, the disclosure relates to a recombinant reverse transcriptase with improved processivity.

### BACKGROUND

Reverse transcriptase (RT) is an enzyme capable of generating a complementary DNA (cDNA) from an RNA template via a process termed reverse transcription. In nature, reverse transcriptase is used by viruses to replicate their genomes, by retrotransposon to proliferate within the host genome, and by eukaryotic cells to extend the telomeres at the ends of the chromosomes. Reverse transcriptase has been widely used in the laboratory to convert RNA to DNA in molecular cloning, RNA sequencing, and reverse transcription polymerase chain reaction. As a result, reverse transcriptase has become an indispensable tool for genetic studies in various fields including biology, medicine and agriculture.

Cold shock proteins are found in various organisms, particularly in microorganisms, to cope with stress and to adapt to changing environment such as downshifting growth temperature, change in pH and salt concentration. Csps are small proteins consisting of 65-80 amino acid residues and their conventionally proposed applications have been limited to their use as a cryoprotective protein which prevents freezing or frost damage in agricultural fields.

Given the importance of reverse transcriptase, some approaches have been made to improve its function. More recently, mutants, fusion proteins and protein mixtures have been created in the quest for improved properties such as thermostability, fidelity and processivity. For example, increasing its processivity by mixing with other nucleic acid-binding proteins such as Ncp7, recA, SSB, T4gp32 (WO2000/055307A2) and cold shock protein (WO2009/108949A2). Despite these improvements that have been made in the processivity of the reverse transcription reaction, synthesizing full-length cDNA for long RNA template remains a problem, and an amount of the synthesized cDNA may not be enough in some cases even today.

### SUMMARY

The improvement of the processivity of the reverse transcription reaction is still an ongoing issue to be desirably further improved, and there is a continuing need to develop new approaches to improve the processivity of reverse transcriptase. Given the limitations of high processivity of reverse transcriptase, there is a need in the art for improved RT compositions and methods to improve upon current techniques.

The present disclosure provides fusion proteins comprising Csps for improved DNA synthesis reactions with improved processivity, methods for synthesizing DNA using such fusion proteins, kits for use in such methods. The fusion proteins, methods and kits disclosed herein address these and other needs.

In one aspect, the present disclosure provides a fusion protein having improved activity of reverse transcription or processivity. In some embodiments, the fusion protein has improved activity of reverse transcription or processivity compared with the protein without the Csp fusion. In some embodiments, the fusion protein has improved processivity that can synthesize long-stranded cDNA of over 15 kb. In some embodiments, the fusion protein can synthesize long-stranded cDNA at lower temperatures. In some embodiments, the fusion protein can synthesize long-stranded cDNA at no more than 50° C. In some embodiments, the fusion protein can synthesize long-stranded cDNA at no more than 42° C. In some embodiments, the fusion protein can reverse transcribe secondary structure-rich RNA at lower temperatures. In some embodiments, the fusion protein can reverse transcribe secondary structure-rich RNA at no more than 50° C. In some embodiments, the fusion protein can reverse transcribe secondary structure-rich RNA at no more than 42° C.

In one aspect, the fusion protein comprises: a cold shock protein (Csp); and a reverse transcriptase (RT) operably linked to the Csp. In some embodiments, the RT is linked to N-terminus of the Csp. In some embodiments, the RT is linked to C-terminus of the Csp.

In some embodiments, the Csp comprises a cold shock domain having at least one of the two ribonucleoprotein (RNP) motifs (also known as nucleic acid binding motifs), RNP1 and RNP2. In some embodiments, the RNP1 has a sequence of X₅-G-X₆-G-X₇-I (SEQ ID NO: 2), wherein X₅ is K, R, S, D, E, N, Q, T, H or Y; X₆ is F, Y, W, L, V, A, I, M, H, R or K; and X₇ is F, Y, W, L, V, A, I, M or H. In some embodiments, the RNP2 has a sequence of X₈-X₉-X₁₀-X₁₁-X₁₂ (SEQ ID NO: 3), wherein X₈ is V, A, I, L, M, F or W; X₉ is F, Y, W, H, Q, L, V, I, M or A; X₁₀ is A, I, L, M, F, V or W; X₁₁ is H, Y, F, L, M, V, I, W, Q or A; and X₁₂ is Y, W, L, F, M, V, I, Q, H or A.

In some embodiment, the cold shock domain has at least optionally one motif, optionally two motifs, optionally three motifs, optionally four motifs, optionally five motifs having one formula selected from the group consisting of: (1) G-X₁-X₂-K-X₃-F-X₄ (SEQ ID NO: 1), (2) X₅-G-X₆-G-X₇-I (SEQ ID NO: 2), (3) X₈-X₉-X₁₀-X₁₁-X₁₂ (SEQ ID NO: 3), (4) X₁₃-X₁₄-X₁₅-X₁₆-X₁₇-X₁₈-X₁₉-X₂₀-X₂₁-X₂₂-X₂₃-X₂₄-X₂₅ (SEQ ID NO: 4), or (5) X₂₆-G-X₂₇-X₂₈-A-X₂₉-X₃₀-X₃₁ (SEQ ID NO: 5), wherein: X₁ is T, I, V, Q, N, L, K or R; X₂ is V, I, L, A or G; X₃ is F, T, Y, H, M or W; X₄ is N, T, S or D; X₅ is K, R, S, D, E, N, Q, T, H or Y; X₆ is F, Y, W, L, V, A, I, M, H, R or K; X₇ is F, Y, W, L, V, A, I, M or H; X₈ is V, A, I, L, M, F or W; X₉ is F, Y, W, H, Q, L, V, I, M or A; X₁₀ is A, I, L, M, F, V or W; X₁₁ is H, Y, F, L, M, V, I, W, Q or A; X₁₂ is Y, W, L, F, M, V, I, Q, H or A; X₁₃ is G, D, N or E; X₁₄ is F, I, L, A or Y; X₁₅ is K, P, Q, E or R; X₁₆ is T, A, E, V or S; X₁₇ is L, P or I; X₁₈ is E, D, T, I, A, F, N or K; X₁₉ is E, Q, T, P, A or D; X₂₀ is G or N; X₂₁ is Q, M, T, L, E or D; X₂₂ is K, R, N, E, S, Q, T, L, V, A or I; X₂₃ is V or I; X₂₄ is E, S, T or Q; X₂₅ is Y or F; X₂₆ is K or R; X₂₇ is P, L, N, Y or A; X₂₈ is Q, K, S, T, A or H; X₂₉ is A, V, T, S, G, R or E; X₃₀ is N, E, K, V, C, R, H, G, D or S; and X₃₁ is V, L or I.

In some embodiments, the cold shock domain has a sequence of G-X₁-X₂-K-X₃-F-X₄-X₅-G-X₆-G-X₇-I-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-G-X₂₇-X₂₈-A-X₂₉-X₃₀-X₃₁ (SEQ ID NO: 6), wherein: X₁ is T, I, V, Q, N, L, K or R; X₂ is V, L, A, G or I; X₃ is F, T, Y, H, M or W; X₄ is N, T, S or D; X₅ is K, R, S, D, E, N, Q, T, H or Y; X₆ is F, Y, W, L, V, A, I, M, H, R or K; X₇ is F, Y, W, L, V, A, I, M or H; X₈ is V, A, I, L, M, F or W; X₉ is F, Y, W, H, Q, L, V, I, M or A; X₁₀ is A, I, L, M, F, V or W; X₁₁ is H, Y, F, L, M, V, I, W, Q or A; X₁₂ is Y, W, L, F, M, V, I, Q, H or A; X₁₃ is G, D, N or E; X₁₄ is F, I, L, A or Y; X₁₅ is K, P, Q, E or R; X₁₆ is T, A, E, V or S; X₁₇ is L, P or I; X₁₈ is E, D, T, I, A, F, N or K; X₁₉ is E, Q, T, P, A or D; X₂₀ is G or N; X₂₁ is Q, M, T, L, E or D; X₂₂ is K, R, N, E, S, Q, T, L, V, A or I; X₂₃ is V or I; X₂₄ is E, S, T or Q; X₂₅ is Y or F; X₂₆ is K or R; X₂₇ is P, L, N, Y or A; X₂₈ is Q, K, S, T, A or H; X₂₉ is A, V, T, S, G, R or E; X₃₀ is N, E, K, V, C, R, H, G, D or S; and X₃₁ is V, L or I.

In some embodiments, the Csp is selected from the group consisting of CspA derived from Escherichia coli (ecCspA), Csp2 derived from Thermus thermophilus (ttCsp2), Csp1 derived from Thermus thermophilus (ttCsp1) (see Tanaka et al., FEBS J. 279 (6): 1014-29 (2012)), CspD derived from Escherichia coli (ecCspD), CspH derived from Escherichia coli (ecCspH), CspD derived from Bordetella bronchiseptica (bbCspD), Csp derived from Actinomadura harenae (ahCsp), Csp derived from Alicyclobacillus dauci (adCsp), CspD derived from Bacillus subtilis (bsCspD) and CspA derived from Mycobacterium tuberculosis (mtCspA). In some embodiments, the Csp has an amino acid sequence selected from SEQ ID NOs: 7-39 or a sequence having at least 10%, 20%, 30%, 40%, 50%, 55%, 60%, 65%, 70%, 75%, 80%, 85%, 90%, 91%, 92%, 93%, 94%, 95%, 96%, 97%, 98% or 99% identity thereto.

In some embodiments, the RT is selected from the group consisting of MMLV RT, AMV RT, HIV RT WDSV RT, RSV RT, ASLV RT, REV-T RT, MAV RT, and RAV RT. In some embodiments, the RT has an amino acid sequence selected from SEQ ID NOs: 40-42 or a sequence having at least 10%, 20%, 30%, 40%, 50%, 55%, 60%, 65%, 70%, 75%, 80%, 85%, 90%, 91%, 92%, 93%, 94%, 95%, 96%, 97%, 98% or 99% identity thereto with the reverse transcriptase activity.

In some embodiments, the RT is a mutated RT. In certain embodiments, the RT is a mutant lacking RNase H activity. In some embodiments, the RT is a mutated MMLV (MMLVmut) having at least one mutation selected from D524N, E562Q, D583N, D653N of SEQ ID NO: 40. In certain embodiments, the RT is a mutant with increased heat resistance. In some embodiments, the RT is a mutated MMLV (MMLVmut) having at least one mutation selected from V129R, T197A, H204R, N249D, M289L, Q291I, T306K, F309N, W313F, Y344F, T420V, L435G, N454K, A644P of SEQ ID NO: 40. In some embodiments, the RT is a mutated MMLV having one mutation selected from the group consisting of T197A, H204R, F309N, W313F, L435G and N454K of SEQ ID NO: 40.

In some embodiments, the RT is selected from the group consisting of the DNA polymerases which have reverse transcriptase activity. In some embodiments, the RT is selected from the group consisting of Tth DNA polymerase, Tfl DNA polymerase, Tfi DNA polymerase, Tma DNA polymerase, Tne DNA polymerase, Z05 DNA polymerase, JDF-3 DNA polymerase, Bst DNA polymerase, CA2 DNA polymerase, Cst DNA DNA polymerase, and Bca DNA polymerase. In some embodiments, the RT has an amino acid sequence selected from SEQ ID NOs: 43-48 or a sequence having at least 10%, 20%, 30%, 40%, 50%, 55%, 60%, 65%, 70%, 75%, 80%, 85%, 90%, 91%, 92%, 93%, 94%, 95%, 96%, 97%, 98% or 99% identity thereto with the reverse transcriptase activity.

In some embodiments, the RT is linked to the Csp via a linker. In some embodiments, the linker has an amino acid sequence selected from G, PG, GSG, or any one of SEQ ID NOs: 49-59.

In some embodiments, the fusion protein disclosed herein has an amino acid sequence selected from SEQ ID NOs: 78-83, 86-92, 94, 96, 98 or a sequence having at least 10%, 20%, 30%, 40%, 50%, 55%, 60%, 65%, 70%, 75%, 80%, 85%, 90%, 91%, 92%, 93%, 94%, 95%, 96%, 97%, 98% or 99% identity thereto.

In some embodiments, the fusion protein has improved activity of reverse transcription processivity.

In another aspect, the present disclosure provides a polynucleotide encoding the fusion protein disclosed herein.

In another aspect, the present disclosure provides a vector comprising the polynucleotide disclosed herein.

In another aspect, the present disclosure provides a recombinant host cell suitable for producing a protein, comprising the polynucleotide disclosed herein.

In another aspect, the present disclosure provides a kit for reverse transcription reaction comprising the fusion protein disclosed herein and a reaction buffer solution. In some embodiments, the kit further comprises a primer.

In another aspect, the present disclosure provides a method of synthesizing a DNA. In one embodiment, the method comprises incubating the fusion protein disclosed herein with an RNA template and a primer under a condition suitable for the fusion protein to perform reverse transcription reaction, thereby synthesizing a DNA strand complementary to the RNA template. In some embodiments, the primer is an oligo (dT) primer, a random sequence primer, or a combination thereof.

### BRIEF DESCRIPTION OF DRAWING

The following drawings form part of the present specification and are included to further demonstrate certain aspects of the present disclosure. The disclosure may be better understood by reference to one or more of these drawings in combination with the detailed description of specific embodiments presented herein.

FIG. 1 illustrates the comparison of Bos6C1 activity to competitors. Reverse transcriptase first strand cDNA synthesis activities at 42° C. for 15′ using lambda RNA ladder (1, 2, 3, 5, 7, 9, and 15 kb) or HCV RNA ladder (1, 2, 3, 5, 7, and 9 kb) as templates.

FIG. 2 illustrates the comparison of reverse transcriptase first strand cDNA synthesis activities at 42° C. for 15′ (left) and for 30′ (right) using lambda RNA ladder (1, 2, 3, 5, 7, 9, and 15 kb) as RNA templates. 15 kb cDNA can be clearly seen for Bos6C1 after 30′.

FIG. 3 illustrates the comparison of reverse transcriptase first strand cDNA synthesis activities at 42° C. for 15′ (left) and for 30′ (right) using HCV RNA ladder (1, 2, 3, 5, 7, and 9 kb) as RNA templates. Bos6C1 performs better than competitors.

FIG. 4 illustrates that the linker length does not affect ttCsp1-MMLVmut activity. Reverse transcriptase first strand cDNA synthesis activities at 42° C. for 15′ using lambda RNA ladder (1, 2, 3, 5, 7, 9, and 15 kb) or HCV RNA ladder (1, 2, 3, 5, 7, and 9 kb) as templates.

FIG. 5 illustrates that ttCsp1 can be fused at either N-terminus or C-terminus of MMLVmut. Reverse transcriptase first strand cDNA synthesis activities at 42° C. for 15′ (left) or 30′ (right) using lambda RNA ladder (1, 2, 3, 5, 7, 9, and 15 kb) (top) or HCV RNA ladder (1, 2, 3, 5, 7, and 9 kb) (bottom) as templates.

FIG. 6 illustrates that ttCsp1 can be fused to HIV RT to improve processivity. Reverse transcriptase first strand cDNA synthesis activities at 42° C. for 30′ using lambda RNA ladder (1, 2, 3, 5, 7, 9, and 15 kb) (left) or HCV RNA ladder (1, 2, 3, 5, 7, and 9 kb) (right) as templates.

FIG. 7 illustrates that different Csp proteins can be used to improve processivity of reverse transcriptase. Reverse transcriptase first strand cDNA synthesis activities at 42° C. for 15′ (left) and 30′ (right) using lambda RNA ladder (1, 2, 3, 5, 7, 9, and 15 kb) as templates. Percentages protein sequence identity of various fusion Csp protein to ttCsp1 are shown in brackets. Even ecCspH which only has 31.9% sequence identity to ttCsp1 functions similarly in improving MMLVmut processivity.

FIG. 8 illustrates that different Csp proteins can be used to improve processivity. Reverse transcriptase first strand cDNA synthesis activities at 42° C. for 15′ (left) and 30′ (right) using HCV RNA ladder (1, 2, 3, 5, 7, and 9 kb) as templates.

FIG. 9 illustrates multiple sequence alignment of various Csp proteins from both mesophilic and thermophilic microorganisms (Clustal Omega 1.2.2). Consensus sequence is shown on top. ttCsp1 sequence is moved to the bottom to show amino acids residue numbers used in mutation study in FIG. 10. Percentage pair-wise sequence identity to ttCsp1 of each Csp protein is shown on the right.

FIG. 10 illustrates the effect of mutations in ttCsp1 predicted to affect its binding to RNA. Reverse transcriptase first strand cDNA synthesis activities at 42° C. for 15′ using lambda RNA ladder (1, 2, 3, 5, 7, 9, and 15 kb) as templates.

FIG. 11 illustrates that free Csp hinders reverse transcription of longer RNA pieces. Reverse transcriptase first strand cDNA synthesis activities at 42° C. for 30′ using lambda RNA ladder (1, 2, 3, 5, 7, 9, and 15 kb) as templates.

FIG. 12 illustrates that free Csp hinders reverse transcription of longer RNA pieces. Reverse transcriptase first strand cDNA synthesis activities at 42° C. for 30′ using HCV RNA ladder (1, 2, 3, 5, 7, and 9 kb) as templates.

FIG. 13 illustrates that eukaryotic Csp-like domains can also be fused to MMLV to improve processivity. Reverse transcriptases first strand cDNA synthesis activities at 42° C. for 15′ with lambda RNA ladder (1, 2, 3, 5, 7, 9 and 15 kb) (left) or HCV RNA ladder (1, 2, 3, 5, 7 and 9 kb) (right) as templates.

FIG. 14 illustrates that ttCsp1 can be fused to DNA polymerases with reverse transcriptase activity (Bst, CA2, and CST) to increase polymerases processivity respect to RNA substrates (0.3 kb, 0.5 kb, and 1 kb). These DNA polymerases by themselves can barely reverse transcribe 0.3 kb RNA substrate at 50° C. for 60′. With ttCsp1 fusion (cBst, cCA2 and cCST), they can easily reverse transcribe to 1 kb under the same condition.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### I. Definition

It is to be understood that both the foregoing general description and the following detailed description are exemplary and explanatory only and are not restrictive of the invention as claimed. In this application, the use of the singular includes the plural unless specifically stated otherwise. In this disclosure, the term “or” is used to mean “and/or” unless explicitly indicated to refer to alternatives only or the alternatives are mutually exclusive. As used herein “another” may mean at least a second or more. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

As used herein, the singular forms “a”, “an” and “the” include plural references unless the context clearly dictates otherwise.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure.

The term “mutant” protein as used herein refers to a protein that has one or more amino acid substitutions, deletions (including truncations) or additions (including deletions) relative to a wild-type. A mutant protein may have less than 100% sequence identity to the amino acid sequence of a naturally occurring protein but may have any amino acid that is at least 80%, at least 85%, at least 90%, at least 95%, at least 97%, at least 98%, or at least 99% identical to the amino acid sequence of the naturally occurring protein.

The term “fusion” protein as used herein refers to a type of protein composed of a plurality of polypeptide components that are unjoined in their naturally occurring state. Fusion proteins may be a combination of two, three or even four or more different proteins. The term polypeptide includes fusion proteins, including, but not limited to, a fusion of two or more heterologous amino acid sequences, a fusion of a polypeptide with: a heterologous targeting sequence, a linker, an immunologically tag, a detectable fusion partner, such as a fluorescent protein, etc., and the like. A fusion protein may have one or more heterologous domains added to the N-terminus, C-terminus, and or the middle portion of the protein. If two parts of a fusion protein are “heterologous”, they are not part of the same protein in its natural state.

The term “Csp” as used herein refers to the cold shock protein. Csp are small proteins consisting of 65-80 amino acid residues that can bind to single-stranded nucleic acids via a highly conserved cold shock domain (CSD), which contains two ribonucleoprotein (RNP) motifs, RNP1 and RNP2, which are also known as nucleic acid binding motifs. It shall be understood that the Csp comprised in the fusion protein disclosed herein can be a naturally-occurred Csp, a mutated version of a naturally-occurred Csp, or a fragment of a naturally-occurred Csp, wherein in each case the Csp (or a mutated version or fragment thereof) binds to single-stranded nucleic acids, which results in an improved processivity of the fusion protein.

The term “RT” as used herein refers to the reverse transcriptase. It shall be understood that the RT comprised in the fusion protein disclosed herein can be a naturally-occurred RT, a mutated version of a naturally-occurred RT, or a fragment of a naturally-occurred RT, wherein in each case the RT (or a mutated version or fragment thereof) is a polypeptide or subunit having reverse transcription activity.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

As used herein, an “isolated” biological component (such as a nucleic acid, peptide or cell) has been substantially separated, produced apart from, or purified away from other biological components or cells of the organism in which the component naturally occurs, i.e., other chromosomal and extrachromosomal DNA and RNA, cells and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

The term “link” as used herein refers to the association via intramolecular interaction, e.g., covalent bonds, metallic bonds, and/or ionic bonding, or inter-molecular interaction, e.g., hydrogen bond or noncovalent bonds.

The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19 (18): 5081 (1991); Ohtsuka et al., J. Biol. Chem. 260 (5): 2605-2608 (1985); and Rossolini et al., Mol. Cell. Probes 8 (2): 91-98 (1994)).

The term “operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of the coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215 (3): 403-410 (1990); Stephen F. et al., Nucleic Acids Res., 25 (17): 3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23 (21): 2947-8 (2007)), and ALIGN or Megalign (DNASTAR) software. A person skilled in the art may use the default parameters provided by the tool or may customize the parameters as appropriate for the alignment, such as for example, by selecting a suitable algorithm.

The term “polypeptide” or “protein” means a string of at least two amino acids linked to one another by peptide bonds. Polypeptides and proteins may include moieties in addition to amino acids (e.g., may be glycosylated) and/or may be otherwise processed or modified. Those of ordinary skill in the art will appreciate that a “polypeptide” or “protein” can be a complete polypeptide chain as produced by a cell (with or without a signal sequence) or can be a functional portion thereof. Those of ordinary skill will further appreciate that a polypeptide or protein can sometimes include more than one polypeptide chain, for example linked by one or more disulfide bonds or associated by other means. The term also includes amino acid polymers in which one or more amino acids are chemical analogs of a corresponding naturally occurring amino acid and polymers.

The term “recombinant” when used with reference to a polypeptide (e.g., antibody, antigen) or a polynucleotide, refers to a polypeptide or polynucleotide that is produced by a recombinant method. A “recombinant polypeptide” includes any polypeptide expressed from a recombinant polynucleotide. A “recombinant polynucleotide” includes any polynucleotide which has been modified by the introduction of at least one exogenous (i.e., foreign, and typically heterologous) nucleotide or the alteration of at least one native nucleotide component of the polynucleotide and need not include all of the coding sequence or the regulatory elements naturally associated with the coding sequence. A “recombinant vector” refers to a non-naturally occurring vector, including, e.g., a vector comprising a recombinant polynucleotide sequence.

As used herein, a “vector” refers to a nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

### II. Fusion Protein and Production Thereof

### A. Fusion Protein

The present disclosure in one aspect provides a fusion protein with improved processivity of reverse transcription, i.e., with improved activity of cDNA synthesis. In one embodiment, the fusion protein disclosed herein comprises a reverse transcriptase (RT) and a cold shock protein (Csp) operably linked to the RT. It is appreciated that the fusion protein of the present disclosure can have various forms and structures. The Csp could link to N-terminus or C-terminus of the RT. The Csp could link to the RT directly or indirectly, e.g., via a linker.

### Cold Shock Protein

As used herein, cold shock proteins (Csps) refer to a group of proteins that are expressed in organisms, particularly in microorganisms, to cope with stress and to adapt to changing environment such as downshifting growth temperature, change in pH and salt concentration. Csps are highly conserved and diverse proteins in terms of structure and function, respectively (see Chaudhary et al., Int J Biol Macromol. 220:743-753 (2022)). Csps are multi-function proteins and they interact with different types of biomolecules, including DNA, RNA, as well as proteins. They play key role in transcriptional regulation and post-translation modifications related to several metabolic pathways. Meanwhile, it also shows that CspA and CspC act the similar function in some reactions (Derman et al., Food Microbiol. 46:463-470 (2014)). In prokaryotes, Csps are involved in various cellular and metabolic processes such as growth and development, osmotic oxidation, starvation, stress tolerance, and host cell invasion. Eukaryotic Csps are evolved form of prokaryotic Csps where cold shock domain is flanked by N- and C-terminal domains. In eukaryotes, Csps can act as nucleic acid chaperons by preventing the formation of secondary structures in mRNA at low temperatures. Furthermore, Csp are small proteins consisting of 65-80 amino acid residues that can bind to single-stranded nucleic acids via a highly conserved cold shock domain (CSD), which contains two ribonucleoprotein (RNP) motifs, RNP1 and RNP2, which are also known as nucleic acid binding motifs (see Tanaka et al., FEBS J. 279 (6): 1014-29 (2012)).

It shall be understood that the Csp comprised in the fusion protein disclosed herein can be a naturally-occurred Csp, a mutated version of a naturally-occurred Csp, or a fragment of a naturally-occurred Csp, wherein in each case the Csp (or a mutated version or fragment thereof) binds to single-stranded nucleic acids, which results in an improved processivity of the fusion protein.

Naturally-occurred Csps can be found in many organisms. For example, Escherichia coli possesses nine Csps (ecCsps), i.e., ecCspA to ecCspI, with ecCspD and ecCspF sharing the lowest sequence identity of 26.9%. ecCspA has been demonstrated to be an RNA chaperon while the functions of other ecCsps are less clear. Csps also exist in other organisms, including Bacillus subtilis (bsCspB), Bacillus caldolyticus (bcCspB), Thermotoga maritima (tmCspB, tmCspL), Neisseria meningitidis (nmCsp), Salmonella typhimurium (stCsp), Thermus thermophilus (ttCsp1, ttCsp2), Actinomadura harenae (ahCsp), Lactobacillus plantarum (IpCspL) and so on. The amino acid sequences of exemplary naturally-occurred Csps are listed in Table 1.

TABLE 1
Examples of Exemplary Cold Shock Proteins/Domains

| | Csp protein/ | | SEQ ID |
|---|---|---|---|
| Organism | domain | Sequence | NO: |
| Actinomadura harenae | ahCsp | MAQGTVKWFNADKGYGFIAVDGGADVFVHYSVIQMD | 7 |
| | | GYRSLEQGQRVEFEITQSDRGPQAESVRLL | |
| Alicyclobacillaceae | abCsp | MQGRVKWFNPDKGYGFISKDDGEDVFVHYSAIQTQGY | 8 |
| bacterium | | RTLEEGQLVEFDIVQGARGPQAANVVPVGP | |
| Alicyclobacillus dauci | adCsp | MQQGTVKWFNGDKGFGFISVEGGEDVFVHFSAIQSNG | 9 |
| | | FRSLDEGQRVEFDIVEGPKGPQAANVVVIR | |
| Alicyclobacillus tolerans | atCsp | MTQGTVKWFNGDKGFGFISVEGGNDVFVHFSAIQSDG | 10 |
| | | FRTLEEGQVVEFEIVEGQRGPQAANVVVIR | |
| Bacillus subtilis | bsCspC | MEQGTVKWFNAEKGFGFIERENGDDVFVHFSAIQSDG | 11 |
| | | FKSLDEGQKVSFDVEQGARGAQAANVQKA | |
| Bacillus subtilis | bsCspD | MQNGKVKWFNNEKGFGFIEVEGGDDVFVHFTAIEGDG | 12 |
| | | YKSLEEGQEVSFEIVEGNRGPQASNVVKL | |
| Bacillus subtilis | bsCspB | MLEGKVKWFNSEKGFGFIEVEGQDDVFVHFSAIQGEGF | 13 |
| | | KTLEEGQAVSFEIVEGNRGPQAANVTKEA | |
| Bordetella pertussis | bpCspA | METGVVKWFNAEKGYGFITPEAGGKDLFAHFSEIQAN | 14 |
| | | GFKSLEENQRVSFVTAMGPKGPQATKIQIL | |
| Escherichia coli | ecCspA | MSGKMTGIVKWFNADKGFGFITPDDGSKDVFVHFSAI | 15 |
| | | QNDGYKSLDEGQKVSFTIESGAKGPAAGNVTSL | |
| Escherichia coli | ecCspB | MSNKMTGLVKWFNADKGFGFISPVDGSKDVFVHFSAI | 16 |
| | | QNDNYRTLFEGQKVTFSIESGAKGPAAANVIITD | |
| Escherichia coli | ecCspC | MAKIKGQVKWFNESKGFGFITPADGSKDVFVHFSAIQG | 17 |
| | | NGFKTLAEGQNVEFEIQDGQKGPAAVNVTAI | |
| Escherichia coli | ecCspD | MEKGTVKWFNNAKGFGFICPEGGGEDIFAHYSTIQMD | 18 |
| | | GYRTLKAGQSVQFDVHQGPKGNHASVIVPVEVEAAVA | |
| Escherichia coli | ecCspE | MSKIKGNVKWFNESKGFGFITPEDGSKDVFVHFSAIQT | 19 |
| | | NGFKTLAEGQR VEFEITNGAKGPSAANVIAL | |
| Escherichia coli | ecCspF | MSRKMTGIVKTFDGKSGKGLITPSDGRIDVQLHVSALN | 20 |
| | | LRDAEEITTGLRVEFCRINGLRGPSAANVYLS | |
| Escherichia coli | ecCspG | MSNKMTGLVKWFNADKGFGFITPDDGSKDVFVHFTAI | 21 |
| | | QSNEFRTLNENQKVEFSIEQGQRGPAAANVVTL | |
| Escherichia coli | ecCspH | MSRKMTGIVKTFDRKSGKGFIIPSDGRKEVQVHISAFTP | 22 |
| | | RDAEVLIPGLRVEFCRVNGLRGPTAANVYLS | |
| Escherichia coli | ecCspI | MSNKMTGLVKWFNPEKGFGFITPKDGSKDVFVHFSAI | 23 |
| | | QSNDFKTLTENQEVEFGIENGPKGPAAVHVVAL | |
| Mycobacterium tuberculosis | mtCspA | MPQGTVKWFNAEKGFGFIAPEDGSADVFVHYTEIQGT | 24 |
| | | GFRTLEENQKVEFEIGHSPKGPQATGVRSL | |
| Mycolicibacterium smegmatis | msCsp | MPQGTVKWFNAEKGFGFIAPEDGSADVFVHYTEIQGS | 25 |
| | | GFRTLEENQKVEFEVGQSPKGPQATGVRTI | |
| Shewanella violacea | svCspA | MSDSNTGTVKWFNEDKGFGFLTQDNGGADVFVHFRAI | 26 |
| | | ASEGFKTLDEGQKVTFEVEQGPKGLQASNVIAL | |
| Tepidamorphus gemmatus | tgCsp | MRQTGTVKFFNQSKGYGFISPDGGGSDVFVHVSDVQR | 27 |
| | | SGIPALDQGMRISYETQPDKRGKGPKAVELQVAG | |
| Tepidamorphus gemmatus | tgemCsp | MRQNGTIKFFNHSRGFGFITPDSGSKDVFVHITALERSG | 28 |
| | | LQAPDEGTKVSFEIEEDRRGRGPQAVNIQLA | |
| Tepidimicrobium xylanilyticum | txCsp | MVNGTVKWFNSEKGFGFIKTEEGNDVFVHYSQINKQG | 29 |
| | | FKTLEEGETVSFRIVQGQKGPQAEDVTPIK | |
| Terrisporobacter | tCsp | MTNGTVKWFNNDKGFGFISVEGGDDVFAHFSAIKSDG | 30 |
| | | YKSLEEGQKVSFDIVQGARGPQAENITIL | |
| Terrisporobacter glycolicus | tglyCsp | MSNGIVKWFNSEKGFGFITVEGGEDVFAHFSAIQTDGY | 31 |
| | | KTLEEGQKVSFNIVKGARGPQAENITIL | |
| Terrisporobacter sp. | tsCsp | MNNGIVKWFNNEKGFGFISVEGGDDVFAHFTAIQGEGF | 32 |
| | | KSLEEGQKVSFDIVEGAKGLQAANITIL | |
| Thermotogota bacterium | tbCsp | MKGTVKWFDPKKGYGFITKEEGGDVFVHWSALEMDG | 33 |
| | | FKTLKDGQEVEFEIQEGPKGPQAAHVKVLS | |
| Thermus thermophilus | ttCsp2 | MNKGIVKWFNAEKGYGFIQQEEGPDVFVHFSAIEAEGF | 34 |
| | | RTLNEGERVEFEVEPGRNGKGPQARRVRRL | |
| Thermus thermophilus | ttCsp1 | MQKGRVKWFNAEKGYGFIEREGDTDVFVHYTAINAK | 35 |
| | | GFRTLNEGDIVTFDVEPGRNGKGPQAVNVTVVEPARR | |
| Vibrio cholerae serotype 01 | vcCspD | MYSMATGTVKWFNNAKGFGFICPEGEDGDIFAHYSTIQ | 36 |
| | | MDGYRTLKAGQQVSYQVEQGPKGYHASCVVPIEGQS | |
| | | AK | |
| Homo sapien | YBX1 | DKKVIATKVLGTVKWFNVRNGYGFINRNDTKEDVFVH | 37 |
| | | QTAIKKNNPRKYLRSVGDGETVEFDVVEGEKGAEAAN | |
| | | VTGP | |
| Homo sapien | YBX2 | ADKPVLATKVLGTVKWFNVRNGYGFINRNDTKEDVF | 38 |
| | | VHQTAIKRNNPRKFLRSVGDGETVEFDVVEGEKGAEA | |
| | | TNVTGPGGVPVKGSRYAPNR | |
| Homo sapien | CSDE1a | YPNGTSAALRETGVIEKLLTSYGFIQCSERQARLFFHCS | 39 |
| | | QYNGNLQDLKVGDDVEFEVSSDRRTGKPIAVKLVKI | |

It should be understood that other naturally occurred Csps can be identified using the methods known in the art, for example, by sequence alignment.

In some embodiments, the Csp comprised in the fusion protein disclosed herein contains one or more mutations, e.g., deletion, insertion or substitution, as compared to a naturally occurred Csp. The inventors have further discovered that insertion of R or S (the native amino acid in tgemCsp, ecCspF and ecCspH) in place of K at position 13 of ttCsp1 also increased the fusion protein's processivity. Thus, in some embodiments, the Csp comprised in the fusion protein disclosed herein is derived from a Thermus species and contains a substitution at K13 of SEQ ID NO: 35. For example, where the Csp is derived from a Thermus species, the amino acid at the position corresponding to position 13 of SEQ ID NO: 35 can be selected from S, A, G, V, L, I, M, F, W, P, T, C, Y, N, Q, D, R, K, E or H. In some embodiments, the amino acid at the position corresponding to position 13 of SEQ ID NO: 35 is selected from K, S, or R. In some embodiments, the amino acid at the position corresponding to position 13 of SEQ ID NO: 35 is K.

Further, the inventors found that other amino acids, located nearby to the amino acid corresponding to position 13 of SEQ ID NO: 35, can also be substituted to produce a fusion protein having increased processivity. For example, substitutions at amino acid corresponding to position 5, 6, 8, 10, 15, 17, 26, 27, 28, 29, 30, 32, 33, 34, 35, 36, 37, 38, 39, 40, 41, 42, 43, 44, 58, 60, 61, 63, 64 and/or 65 of SEQ ID NO: 35 also produce a fusion protein having increased processivity as well as the control fusion protein.

In certain embodiments, the Csp is a mutated Csp having at least one mutation selected from the group consisting of K13A, Y15A, F17A, F27A, H29A, Y30A, F38A, K58A of SEQ ID NO: 35.

In some embodiments, the Csp comprised in the fusion protein disclosed herein is a fragment of a naturally occurred Csp protein. In some embodiments, the Csp comprised in the fusion protein disclosed herein contains the cold shock domain (CSD). In some embodiments, the Csp comprised in the fusion protein disclosed herein contains at least one of the two ribonucleoprotein (RNP) motifs (also known as nucleic acid binding motifs), RNP1 and RNP2.

In some embodiments, the RNP1 has at least one sequence of X₅-G-X₆-G-X₇-I (SEQ ID NO: 2), wherein X₅ is K, R, S, D, E, N, Q, T, H or Y; X₆ is F, Y, W, L, V, A, I, M, H, R or K; and X₇ is F, Y, W, L, V, A, I, M or H.

In some embodiments, the RNP2 has at least one sequence of X₈-X₉-X₁₀-X₁₁-X₁₂ (SEQ ID NO: 3), wherein X₈ is V, A, I, L, M, F or W; X₉ is F, Y, W, H, Q, L, V, I, M or A; X₁₀ is A, I, L, M, F, V or W; X₁₁ is H, Y, F, L, M, V, I, W, Q or A; and X₁₂ is Y, W, L, F, M, V, I, Q, H or A.

In some embodiments, the cold shock domain has at least one sequence of G-X₁-X₂-K-X₃-F-X₄ (SEQ ID NO: 1), wherein X₁ is T, I, V, Q, N, L, K or R; X₂ is V, I, L, A or G; X₃ is F, T, Y, H, M or W; X₄ is N, T, S or D.

In some embodiments, the cold shock domain has at least one sequence of X₁₃-X₁₄-X₁₅-X₁₆-X₁₇-X₁₈-X₁₉-X₂₀-X₂₁-X₂₂-X₂₃-X₂₄-X₂₅ (SEQ ID NO: 4), wherein X₁₃ is G, D, N or E; X₁₄ is F, I, L, A or Y; X₁₅ is K, P, Q, E or R; X₁₆ is T, A, E, V or S; X₁₇ is L, P or I; X₁₈ is E, D, T, I, A, F, Nor K; X₁₉ is E, Q, T, P, A or D; X₂₀ is G or N; X₂₁ is Q, M, T, L, E or D; X₂₂ is K, R, N, E, S, Q, T, L, V, A or I; X₂₃ is V or I; X₂₄ is E, S, T or Q; X₂₅ is Y or F.

In some embodiments, the cold shock domain has at least one sequence of X₂₆-G-X₂₇-X₂₈-A-X₂₉-X₃₀-X₃₁ (SEQ ID NO: 5), wherein X₂₆ is K or R; X₂₇ is P, L, N, Y or A; X₂₈ is Q, K, S, T, A or H; X₂₉ is A, V, T, S, G, R or E; X₃₀ is N, E, K, V, C, R, H, G, D or S; and X₃₁ is V, L or I.

In some embodiments, the cold shock domain has a sequence of G-X₁-X₂-K-X₃-F-X₄-X₅-G-X₆-G-X₇-I-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-G-X₂₇-X₂₈-A-X₂₉-X₃₀-X₃₁ (SEQ ID NO: 6), wherein: X₁ is T, I, V, Q, N, L, K or R; X₂ is V, I, L, A or G; X₃ is F, T, Y, H, M or W; X₄ is N, T, S or D; X₅ is K, R, S, D, E, N, Q, T, H or Y; X₆ is F, Y, W, L, V, A, I, M, H, R or K; X₇ is F, Y, W, L, V, A, I, M or H; X₈ is V, A, I, L, M, F or W; X₉ is F, Y, W, H, Q, L, V, I, M or A; X₁₀ is A, I, L, M, F, V or W; X₁₁ is H, Y, F, L, M, V, I, W, Q or A; X₁₂ is Y, W, L, F, M, V, I, Q, H or A; X₁₃ is G, D, N or E; X₁₄ is F, I, L, A or Y; X₁₅ is K, P, Q, E or R; X₁₆ is T, A, E, V or S; X₁₇ is L, P or I; X₁₈ is E, D, T, I, A, F, Nor K; X₁₉ is E, Q, T, P, A or D; X₂₀ is G or N; X₂₁ is Q, M, T, L, E or D; X₂₂ is K, R, N, E, S, Q, T, L, V, A or I; X₂₃ is V or I; X₂₄ is E, S, T or Q; X₂₅ is Y or F; X₂₆ is K or R; X₂₇ is P, L, N, Y or A; X₂₈ is Q, K, S, T, A or H; X₂₉ is A, V, T, S, G, R or E; X₃₀ is N, E, K, V, C, R, H, G, D or S; and X₃₁ is V, L or I.

In some embodiments, X₁ is I, T or R. In some embodiments, X₁ is I. In some embodiments, X₁ is T. In some embodiments, X₁ is R. In some embodiments, X₂ is V, L, A or G. In some embodiments, X₂ is V. In some embodiments, X₂ is L. In some embodiments, X₂ is A. In some embodiments, X₂ is G. In some embodiments, X₃ is T, Y, H, M or W. In some embodiments, X₃ is T. In some embodiments, X₃ is Y. In some embodiments, X₃ is H. In some embodiments, X₃ is M. In some embodiments, X₃ is W. In some embodiments, X₄ is D, T, S or N. In some embodiments, X₄ is D. In some embodiments, X₄ is T. In some embodiments, X₄ is S. In some embodiments, X₄ is N. In some embodiments, X₅ is S or K. In some embodiments, X₅ is S. In some embodiments, X₅ is K. In some embodiments, X₆ is K, F or Y. In some embodiments, X₆ is K. In some embodiments, X₆ is F. In some embodiments, X₆ is Y. In some embodiments, X₇ is I. In some embodiments, X₈ is V or I. In some embodiments, X₈ is V. In some embodiments, X₈ is I. In some embodiments, X₉ is Q or F. In some embodiments, X₉ is Q. In some embodiments, X₉ is F. In some embodiments, X₁₀ is V or A. In some embodiments, X₁₀ is V. In some embodiments, X₁₀ is A. In some embodiments, X₁₁ is H. In some embodiments, X₁₂ is I, F or Y. In some embodiments, X₁₂ is I. In some embodiments, X₁₂ is F. In some embodiments, X₁₂ is Y. In some embodiments, X₁₃ is D or G. In some embodiments, X₁₃ is D. In some embodiments, X₁₃ is G. In some embodiments, X₁₄ is A, Y or F. In some embodiments, X₁₄ is A. In some embodiments, X₁₄ is Y. In some embodiments, X₁₄ is F. In some embodiments, X₁₅ is E, K or R. In some embodiments, X₁₅ is E. In some embodiments, X₁₅ is K. In some embodiments, X₁₅ is R. In some embodiments, X₁₆ is V, S or T. In some embodiments, X₁₆ is V. In some embodiments, X₁₆ is S. In some embodiments, X₁₆ is T. In some embodiments, X₁₇ is L. In some embodiments, X₁₈ is T, D, K, E or N. In some embodiments, X₁₈ is T. In some embodiments, X₁₈ is D. In some embodiments, X₁₈ is K. In some embodiments, X₁₈ is E. In some embodiments, X₁₈ is N. In some embodiments, X₁₉ is P, E, A, Q or E. In some embodiments, X₁₉ is P. In some embodiments, X₁₉ is E. In some embodiments, X₁₉ is A. In some embodiments, X₁₉ is Q. In some embodiments, X₁₉ is E. In some embodiments, X₂₀ is G. In some embodiments, X₂₁ is L, Q or D. In some embodiments, X₂₁ is L. In some embodiments, X₂₁ is Q. In some embodiments, X₂₁ is D. In some embodiments, X₂₂ is R, S or I. In some embodiments, X₂₂ is R. In some embodiments, X₂₂ is S. In some embodiments, X₂₂ is I. In some embodiments, X₂₃ is V. In some embodiments, X₂₄ is E, S, Q or T. In some embodiments, X₂₄ is E. In some embodiments, X₂₄ is S. In some embodiments, X₂₄ is Q. In some embodiments, X₂₄ is T. In some embodiments, X₂₅ is F. In some embodiments, X₂₆ is K or R. In some embodiments, X₂₆ is K. In some embodiments, X₂₆ is R. In some embodiments, X₂₇ is P or N. In some embodiments, X₂₇ is P. In some embodiments, X₂₇ is N. In some embodiments, X₂₈ is T, A, H or Q. In some embodiments, X₂₈ is T. In some embodiments, X₂₈ is A. In some embodiments, X₂₈ is H. In some embodiments, X₂₈ is Q. In some embodiments, X₂₉ is A, G, S, E or V. In some embodiments, X₂₉ is A. In some embodiments, X₂₉ is G. In some embodiments, X₂₉ is S. In some embodiments, X₂₉ is E. In some embodiments, X₂₉ is V. In some embodiments, X₃₀ is N, V or S. In some embodiments, X₃₀ is N. In some embodiments, X₃₀ is V. In some embodiments, X₃₀ is S. In some embodiments, X₃₁ is V or I. In some embodiments, X₃₁ is V. In some embodiments, X₃₁ is I.

### Reverse Transcriptase

Reverse transcriptase (RT), also known as RNA-dependent DNA polymerase, is a DNA polymerase enzyme that synthesize DNA complementary to RNA used as a template. Any reverse transcriptase can be used in the present invention as far as it has a reverse transcription activity. Examples of the reverse transcriptase include those derived from viruses such as a reverse transcriptase derived from Moloney Murine Leukemia Virus (reverse transcriptase derived from MMLV), a reverse transcriptase derived from Avian Myeloblastosis Virus (reverse transcriptase derived from AMV), a reverse transcriptase derived from Human Immunodeficiency Virus (reverse transcriptase derived from HIV), a reverse transcriptase derived from Rous Sarcoma Virus (reverse transcriptase derived from RSV), a reverse transcriptase derived from Walleye Dermal Sarcoma Virus (reverse transcriptase derived from WDSV), a reverse transcriptase derived from Avian Sarcoma-Leukosis Virus (reverse transcriptase derived from ASLV), a reverse transcriptase derived from Avian Reticuloendotheliosis Virus (reverse transcriptase derived from REV-T), a reverse transcriptase derived from Myeloblastosis Associated Virus (reverse transcriptase derived from MAV), a reverse transcriptase derived from Rous Associated Virus (reverse transcriptase derived from RAV), and reverse transcriptases derived from eubacteria such as DNA polymerase derived from bacterium of the genus Thermus thermophiles (Tth DNA polymerase, and the like), DNA polymerase derived from bacterium of the genus Thermus filiformis (Tfi DNA polymerase, and the like), DNA polymerase derived from bacterium of the genus Thermus flavus (Tfl DNA polymerase, and the like), DNA polymerase derived from bacterium of the genus Thermotoga maritima (Tma DNA polymerase, and the like), DNA polymerase derived from bacterium of the genus Thermotoga neapolitana (Tne DNA polymerase, and the like), DNA polymerase derived from bacterium of the genus Thermus species Z05 (Z05 DNA polymerase, and the like), DNA polymerase derived from bacterium of the genus Thermococcus species JDF-3 (JDF-3 DNA polymerase, and the like), DNA polymerase derived from thermophilic bacterium of the genus Bacillus stearothermophilus (Bst DNA polymerase, and the like) and DNA polymerase derived from thermophilic bacterium of the genus Bacillus caldotenax (Bca DNA polymerase, and the like). The reverse transcriptases derived from viruses are preferably used, and the reverse transcriptase derived from MMLV, AMV or HIV is more preferably used in the present invention. Further, a reverse transcriptase modified into a naturally-derived amino acid sequence can also be used in the present invention as far as it has the reverse transcription activity. The amino acid sequences of exemplary reverse transcriptases are listed in Table 2.

TABLE 2
Examples of Exemplary Reverse Transcriptases

| Reverse transcriptase protein | Sequence | SEQ ID NO: |
|---|---|---|
| MMLV Reverse Transcriptase | LNIEDEHRLHETSKEPDVSLGSTWLSDFPQ | 40 |
| | AWAETGGMGLAVRQAPLIIPLKATSTPVSI | |
| | KQYPMSQEARLGIKPHIQRLLDQGILVPCQ | |
| | SPWNTPLLPVKKPGTNDYRPVQDLREVNKR | |
| | VEDIHPTVPNPYNLLSGLPPSHQWYTVLDL | |
| | KDAFFCLRLHPTSQPLFAFEWRDPEMGISG | |
| | QLTWTRLPQGFKNSPTLFDEALHRDLADFR | |
| | IQHPDLILLQYVDDLLLAATSELDCQQGTR | |
| | ALLQTLGNLGYRASAKKAQICQKQVKYLGY | |
| | LLKEGQRWLTEARKETVMGQPTPKTPRQLR | |
| | EFLGTAGFCRLWIPGFAEMAAPLYPLTKTG | |
| | TLFNWGPDQQKAYQEIKQALLTAPALGLPD | |
| | LTKPFELFVDEKQGYAKGVLTQKLGPWRRP | |
| | VAYLSKKLDPVAAGWPPCLRMVAAIAVLTK | |
| | DAGKLTMGQPLVILAPHAVEALVKQPPDRW | |
| | LSNARMTHYQALLLDTDRVQFGPVVALNPA | |
| | TLLPLPEEGLQHNCLDILAEAHGTRPDLTD | |
| | QPLPDADHTWYTDGSSLLQEGQRKAGAAVT | |
| | TETEVIWAKALPAGTSAQRAELIALTQALK | |
| | MAEGKKLNVYTDSRYAFATAHIHGEIYRRR | |
| | GLLTSEGKEIKNKDEILALLKALFLPKRLS | |
| | IIHCPGHQKGHSAEARGNRMADQAARKAAI | |
| | TETPDTSTLLI | |
| HIV Reverse Transcriptase | PISPIETVPVKLKPGMDGPKVKQWPLTEEK | 41 |
| | IKALVEICTEMEKEGKISKIGPENPYNTPV | |
| | FAIKKKDGTKWRKLVDFRELNKKTQDFWEV | |
| | QLGIPHPAGLKKKKSVTVLDVGDAYFSVPL | |
| | DEDFRKYTAFTIPSINNETPGIRYQYNVLP | |
| | QGWKGSPAIFQSSMTKILEPFRKQNPDIVI | |
| | YQYMDDLYVGSDLEIGQHRTKIEELRQHLL | |
| | RWGLTTPDKKHQKEPPFLWMGYELHPDKWT | |
| | VQPIVLPEKDSWTVNDIQKLVGKLNWASQI | |
| | YPGIKVRQLCKLLRGTKALTEVIPLTEEAE | |
| | LELAENREILKEPVHGVYYDPSKDLIAEIQ | |
| | KQGQGQWTYQIYQEPFKNLKTGKYARMRGA | |
| | HTNDVKQLTEAVQKITTESIVIWGKTPKFK | |
| | LPIQKETWETWWTEYWQATWVPEWEFVNTP | |
| | PLVKLWYQLEKEPIVGAETFYVDGAASRET | |
| | KLGKAGYVTNKGRQKVVTLTDTTNQKTELQ | |
| | AIHLALQDSGLEVNIVTNSQYALGIIQAQP | |
| | DQSESELVNQIIEQLIKKEKVYLAWVPAHK | |
| | GIGGNEQVDKLVSAGIRKIL | |
| AMV Reverse Transcriptase | TVALHLAIPLKWKPNHTPVWIDQWPLPEGK | 42 |
| | LVALTQLVEKELQLGHIEPSLSCWNTPVFV | |
| | IRKASGSYRLLHDLRAVNAKLVPFGAVQQG | |
| | APVLSALPRGWPLMVLDLKDCFFSIPLAEQ | |
| | DREAFAFTLPSVNNQAPARRFQWKVLPQGM | |
| | TCSPTICQLIVGQILEPLRLKHPSLRMLHY | |
| | MDDLLLAASSHDGLEAAGEEVISTLERAGF | |
| | TISPDKVQREPGVQYLGYKLGSTYVAPVGL | |
| | VAEPRIATLWDVQKLVGSLQWLRPALGIPP | |
| | RLMGPFYEQLRGSDPNEAREWNLDMKMAWR | |
| | EIVQLSTTAALERWDPALPLEGAVARCEQG | |
| | AIGVLGQGLSTHPRPCLWLFSTQPTKAFTA | |
| | WLEVLTLLITKLRASAVRTFGKEVDILLLP | |
| | ACFREDLPLPEGILLALRGFAGKIRSSDTP | |
| | SIFDIARPLHVSLKVRVTDHPVPGPTVFTD | |
| | ASSSTHKGVVVWREGPRWEIKEIADLGASV | |
| | QQLEARAVAMALLLWPTTPTNVVTDSAFVA | |
| | KMLLKMGQEGVPSTAAAFILEDALSQRSAM | |
| | AAVLHVRSHSEVPGFFTEGNDVADSQATFQ | |
| | AYPLREAKDLHTALHIGPRALSKACNISMQ | |
| | QAREVVQTCPHCNSAPALEAGVNPRGLGPL | |
| | QIWQTDFTLEPRMAPRSWLAVTVDTASSAI | |
| | VVTQHGRVTSVAAQHHWATAIAVLGRPKAI | |
| | KTDNGSCFTSKSTREWLARWGIAHTTGIPG | |
| | NSQGQAMVERANRLLKDKIRVLAEGDGFMK | |
| | RIPTSKQGELLAKAMYALNHFERGENTKTP | |
| | IQKHWRPTVLTEGPPVKIRIETGEWEKGWN | |
| | VLVWGRGYAAVKNRDTDKVIWVPSRKVKPD | |
| | IAQKDEVTKKDEASPLFA | |
| Tth DNA polymerase | MEAMLPLFEPKGRVLLVDGHHLAYRTFFAL | 43 |
| | KGLTTSRGEPVQAVYGFAKSLLKALKEDGY | |
| | KAVFVVFDAKAPSFRHEAYEAYKAGRAPTP | |
| | EDFPRQLALIKELVDLLGFTRLEVPGYEAD | |
| | DVLATLAKKAEKEGYEVRILTADRDLYQLV | |
| | SDRVAVLHPEGHLITPEWLWEKYGLRPEQW | |
| | VDFRALVGDPSDNLPGVKGIGEKTALKLLK | |
| | EWGSLENLLKNLDRVKPENVREKIKAHLED | |
| | LRLSLELSRVRTDLPLEVDLAQGREPDREG | |
| | LRAFLERLEFGSLLHEFGLLEAPAPLEEAP | |
| | WPPPEGAFVGFVLSRPEPMWAELKALAACR | |
| | DGRVHRAADPLAGLKDLKEVRGLLAKDLAV | |
| | LASREGLDLVPGDDPMLLAYLLDPSNTTPE | |
| | GVARRYGGEWTEDAAHRALLSERLHRNLLK | |
| | RLEGEEKLLWLYHEVEKPLSRVLAHMEATG | |
| | VRRDVAYLQALSLELAEEIRRLEEEVFRLA | |
| | GHPFNLNSRDQLERVLFDELRLPALGKTQK | |
| | TGKRSTSAAVLEALREAHPIVEKILQHREL | |
| | TKLKNTYVDPLPSLVHPRTGRLHTRFNQTA | |
| | TATGRLSSSDPNLQNIPVRTPLGQRIRRAF | |
| | VAEAGWALVALDYSQIELRVLAHLSGDENL | |
| | IRVFQEGKDIHTQTASWMFGVPPEAVDPLM | |
| | RRAAKTVNFGVLYGMSAHRLSQELAIPYEE | |
| | AVAFIERYFQSFPKVRAWIEKTLEEGRKRG | |
| | YVETLFGRRRYVPDLNARVKSVREAAERMA | |
| | FNMPVQGTAADLMKLAMVKLFPRLREMGAR | |
| | MLLQVHDELLLEAPQARAEEVAALAKEAME | |
| | KAYPLAVPLEVEVGMGEDWLSAKG | |
| Tfl DNA polymerase | MAMLPLFEPKGRVLLVDGHHLAYRTFFALK | 44 |
| | GLTTSRGEPVQAVYGFAKSLLKALKEDGDV | |
| | VVVVFDAKAPSFRHEAYEAYKAGRAPTPED | |
| | FPRQLALIKELVDLLGLVRLEVPGFEADDV | |
| | LATLAKRAEKEGYEVRILTADRDLYQLLSE | |
| | RIAILHPEGYLITPAWLYEKYGLRPEQWVD | |
| | YRALAGDPSDNIPGVKGIGEKTAQRLIREW | |
| | GSLENLFQHLDQVKPSLREKLQAGMEALAL | |
| | SRKLSKVHTDLPLEVDFGRRRTPNLEGLRA | |
| | FLERLEFGSLLHEFGLLEGPKAAEEAPWPP | |
| | EGAFLGFSFSRPEPMWAELLALAGAWEGRL | |
| | FRAQDPLRGLRDLKGVRGILAKDLAVLALR | |
| | EGLDLFPEDDPMLLAYLLDPSNTTPEGVAR | |
| | RYGGEWTEDAGERALLAERLFQTLKERLYG | |
| | EERLLWLYEEVEKPLSRVLARMEATGVRLD | |
| | VAYLQALSLEVEAEVRQLEEEVFRLAGHPF | |
| | NLNSRDQLERVLFDELGLPAIGKTEKTGKR | |
| | STSAAVLEALREAHPIVDRILQYRELTKLK | |
| | NTYIDPLPALVHPKTGRLHRRFNQTATTGR | |
| | LSSSDPNLQNIPVRTVLGQRIRRAFVAEEG | |
| | WVLVVLDYSQIERLVLAHLSGTENLIRVFQ | |
| | EGREIHTQTASWMFGVSPEGVDPLMRRAAK | |
| | TINFGVLYGMSAHRLSGELSIPYEEAVAFI | |
| | ERYFQSYPKVRAWIEGTLEEGRRRGYVETL | |
| | FGRRRYVPDLNARVKSVREAAERMAFNNPV | |
| | QGAADLMKLAMVRLFPRLQELGARMLLQVH | |
| | DELVLEAPKDRAERVAALAKEVMEGVWPLQ | |
| | VPLEVEVGLGEDWLSAKE | |
| Tfi DNA polymerase | MTPLFDLEEPPKRVLLVDGHHLAYRTFYAL | 45 |
| | SLTTSRGEPVQMVYGFARSLLKALKEDGQA | |
| | VVVVFDAKAPSFRHEAYEAYKAGRAPTPED | |
| | FPRQLALVKRLVDLLGLVRLEAPGYEADDV | |
| | LGTLAKKAEREGMEVRILTGDRDFFQLLSE | |
| | KVSVLLPDGTLVTPKVQEKYGVPPERWVDF | |
| | RALTGDRSDNIPGVAGIGEKTALRLLAEWG | |
| | SVENLLKNLDRVKPDSVRRKIEAHLEDLRL | |
| | SLDLARIRTDLPLEVDFKALRRRTPDLEGL | |
| | RAFLEELEFGSLLHEFGLLGGEKPREEAPW | |
| | PPPEGAFVGFLLSRKEPMWAELLALAAAAE | |
| | GRVHRATSPVEALADLKEARGFLAKDLAVL | |
| | ALREGVALDPTDDPLLVAYLLDPANTNPEG | |
| | VARRYGGEFTEDAAERALLSERLFQNLFER | |
| | LSEKLLWLYQEVERPLSRVLAHMEARGVRL | |
| | DVPLLEALSFELEKEMERLEGEVFRLAGHP | |
| | FNLNSRDQLERVLFDELGLTPVGRTEKTGR | |
| | STAQGALEALRGAHPIVELILQYRELSKLK | |
| | STYLDPLPRLVHPRTGRLHTRFNQTATATG | |
| | RLSSSTPNLQNIPVRTPLGQRIRKAFVAEE | |
| | GWLLLAADYSQIELRVLAHLSGDENLKRVF | |
| | REGKDIHTETAAWMFGLDPALVDPKMRRAA | |
| | KTVNFGVLYGMSAHRLSQELGIDYKEAEAF | |
| | IERYFQSFPKVRAWIERTLEEGRTRGYVET | |
| | LFGRRRYVPDLASRVRSVREAAERMAFNMP | |
| | VQGTAADLMKIAMVKLFPRLKPLGAHLLLQ | |
| | VHDELVLEVPEDRAEEAKALVKEVMENTYP | |
| | LDVPLEVEVGVGRDWLEAKGD | |
| Bst DNA polymerase | AFTLADRVTEEMLADKAALVVEVVEENYHD | 46 |
| | APIVGIAVVNEHGRFFLRPETALADPQFVA | |
| | WLGDETKKKSMFDSKRAAVALKWKGIELCG | |
| | VSFDLLLAAYLLDPAQGVDDVAAAAKMKQY | |
| | EAVRPDEAVYGKGAKRAVPDEPVLAEHLVR | |
| | KAAAIWALERPFLDELRRNEQDRLLVELEQ | |
| | PLSSILAEMEFAGVKVDTKRLEQMGEELAE | |
| | QLRTVEQRIYELAGQEFNINSPKQLGVILF | |
| | EKLQLPVLKKTKTGYSTSADVLEKLAPYHE | |
| | IVENILHYRQLGKLQSTYIEGLLKVVRPDT | |
| | KKVHTIFNQALTQTGRLSSTEPNLQNIPIR | |
| | LEEGRKIRQAFVPSESDWLIFAADYSQIEL | |
| | RVLAHIAEDDNLMEAFRRDLDIHTKTAMDI | |
| | FQVSEDEVTPNMRRQAKAVNFGIVYGISDY | |
| | GLAQNLNISRKEAAEFIERYFESFPGVKRY | |
| | MENIVQEAKQKGYVTTLLHRRRYLPDITSR | |
| | NFNVRSFAERMAMNTPIQGSAADIIKKAMI | |
| | DLNARLKEERLQARLLLQVHDELILEAPKE | |
| | EMERLCRLVPEVMEQAVTLRVPLKVDYHYG | |
| | STWYDAK | |
| CA2 DNA polymerase | EGVDVRCPDRPEEVEEALSRLEAAQSVVVE | 47 |
| | VTGDNPHDGEVRGVAWWDGHTAYFIPFERL | |
| | VQSDMRPLADWLADARRPKRTHDSHRAEVA | |
| | LFWHGLAFRGTSFCTHIAAYLLDPTESRHT | |
| | LADLSRRYGLPPVPEAEDVYGKGAKFKVPD | |
| | RDTLARYVGRKAALVARLVPLLEADLAACG | |
| | MRSLFYDLELPLSSELAVMETVGVRVDAAA | |
| | LAAYGEELREAAAKVEREIYELAGTTFNIG | |
| | STKQLGEILFDKLGLPVVKKTKTGYSTDAD | |
| | VLEELAPYHPIVEKILHYRQLTKLQSTYIE | |
| | GLLKEIRPQTGKIHTYYQQTIAATGRLSSQ | |
| | FPNLQNIPIRLEEGRKIRKAFVPSEPGWLM | |
| | LAADYSQIELRVLAHVSGDERLKEAFRTGM | |
| | DIHTKTAMDVFGVSEDRVDARMRRQAKAVN | |
| | FGIIYGISDFGLAQNLNISRKEAAEFIRQY | |
| | FAVFSGVKAYRERIVEQARRDGYVTTLLGR | |
| | RRYLPDINASNYNLRSFAERTAMNTPIQGT | |
| | AADIIKTAMVRLTRRMRDVGLKSRMLLQVH | |
| | DELVFEVPPDELDAMRELVTDVMESAVPLD | |
| | VPLKVDVSWGADWYAAK | |
| Cst DNA polymerase | ELKITHISAAEDLKKWIAYLLNQKNISVLQ | 48 |
| | LIDREDSYSSRLSGLALCTGDEVFYIETGT | |
| | ALPENLIATELKELWQNENIHKIGHNIKEF | |
| | ITWLLKHDVELNGLYFDTMIAEYLIDSIRN | |
| | GYPIASLSHKYLNRSVPSLDELLGKGKGAK | |
| | KYSEIPPERLKDYSAYNVKAIFDIWPMQKK | |
| | VLQENRQEELFNDIELPLITVLASMEYHGF | |
| | KVDAAKLHEYGEVLLSRIKDLEKVIYMLAG | |
| | EEFNINSTKQLGTILFEKLKLPVVKSTKTG | |
| | YSTDVEVLEELYYKHDIIPCIIEYRQLTKL | |
| | YTTYAEGLEKVINPVTGKIHSSFNQTVTAT | |
| | GRISSTEPNLQNIPVRHEMGREIRKAFIPS | |
| | SENAVFVDADYSQIELRVLAHITGDEALIN | |
| | AFVKGEDIHTATASLVFDVAPEDVTPELRR | |
| | KAKAVNFGIVYGISDYGLARDLGITRKEAK | |
| | RYIDDYFAKYPKVKTYVDEIVRVGQEQGYV | |
| | ETLFHRRRYLPELASKNFHQRSFGKRVAMN | |
| | TPIQGTAADIIKIAMVKVYKALKESGLKSR | |
| | LILQVHDELVIETFEDELETVKELVKKCME | |
| | EAVELSVPLVVDVSIGKNWYEAS | |

It shall be understood that the RT comprised in the fusion protein disclosed herein can be a naturally-occurred RT, a mutated version of a naturally-occurred RT, or a fragment of a naturally-occurred RT, wherein in each case the RT (or a mutated version or fragment thereof) is a polypeptide or subunit having reverse transcription activity.

In certain embodiments, the RT is a mutant lacking RNase H activity. In some embodiments, the RT is a mutated MMLV (MML Vmut) having at least one mutation selected from D524N, E562Q, D583N, D653N of SEQ ID NO: 40. In certain embodiments, the RT is a mutant with increased heat resistance. In some embodiments, the RT is a mutated MMLV (MMLVmut) having at least one mutation selected from V129R, T197A, H204R, N249D, M289L, Q291I, T306K, F309N, W313F, Y344F, T420V, L435G, N454K, A644P of SEQ ID NO: 40. In some embodiments, the RT is a mutated MMLV having one mutation selected from the group consisting of T197A, H204R, F309N, W313F, L435G and N454K of SEQ ID NO: 40.

In the exemplary embodiments disclosed in detail herein, the cold shock proteins and reverse transcriptases of the invention are wild or mutant forms of wild-type ttCsp1, ecCspA, ecCspD, ecCspH, ahCsp, MMLV reverse transcriptase or HIV reverse transcriptase, which have altered features that provide the fusion reverse transcriptases with advantageous properties. However, it is to be understood that the invention is not limited to the exemplary embodiments disclosed in detail herein. For example, the invention includes mutants of cold shock proteins other than ttCsp1, such as mutants of any cold shock protein family; or the invention includes mutants of reverse transcriptases other than MMLV reverse transcriptase, such as mutants of any reverse transcriptase derived from virus. These wild-types or mutants can be wild-types or mutants of Csps or reverse transcriptases, including but not limited to those, from species of Thermus thermophilus or Bacillus stearothermophilus. It is well documented and well understood by those of skill in the art that Csps or reverse transcriptases show medium levels of sequence identity and conservation. Thus, it is a simple matter for one of skill in the art to identify similar domains of one particular Csp or reverse transcriptase that correspond to domains of another. Thus, reference herein to specific domains in wild-type Csp or reverse transcriptase can easily be correlated to corresponding domains in other Csp or reverse transcriptase.

### Linker

In certain embodiments, the fusion protein contains a linker that links the Csp to the RT. In certain embodiment, the linkers generally are comprised of helix- and turn-promoting amino acid residues such as alanine, serine and glycine. However, other residues can function as well. In certain embodiments, the linker comprising the amino acid sequence (GGGGS)n or (GSGGS)n (n=2-5). The amino acid sequences of exemplary linkers are listed in Table 3.

TABLE 3
Examples of Exemplary Linkers

| Linker | SEQ ID NO: |
|---|---|
| G | / |
| GSG | / |
| GSGGS | 49 |
| GGGGS | 50 |
| GSGSGSGS | 51 |
| GSGGSGSGGS | 52 |
| GSGGSGSGGSGSGGS | 53 |
| GQGQGQGQGQG | 54 |
| HHHHPGGSVKKR | 55 |
| GSIEGR | 56 |
| SAPGTP | 57 |
| SAPGTPSR | 58 |
| EGKSSGSGSESKEF | 59 |
| PG | / |

While exemplary embodiments discussed in detail herein relate to ttCsps1, other cold shock proteins, reverse transcriptases and other reverse transcriptases derived from other virus, it is to be understood that the mutant Csps or reverse transcriptases may be derived from any Csp or reverse transcriptase having identity to a Csp or reverse transcriptase derived from a virus, a Eubacterial or an Archaeal. Where the mutant Csp or reverse transcriptase is not derived from ttCsp1 or MMLV reverse transcriptase, the mutant Csp or reverse transcriptase can have one or more mutations at domains corresponding to the domains identified herein with specific reference to ttCsp1 or MMLV reverse transcriptase. As will be recognized by those of skill in the art, the Csps or reverse transcriptases may be any cold shock protein or reverse transcriptase derived from virus, Eubacterial or Archaeal, including, but not limited to virus reverse transcriptases, Eubacterial or Archaeal cold shock proteins, as well as mutants or derivatives thereof. Thus, in embodiments, the Csp is derived from a Eubacterial Csp and the reverse transcriptase is derived from virus reverse transcriptase. Suitable Csps or reverse transcriptases can be derived from a variety of thermophilic Eubacteria or virus, including, but not necessarily limited to, Avian Myeloblastosis Virus, Rous Sarcoma Virus, Thermus species and Thermotoga maritima, such as Thermus thermophilus (Tth), and Thermotoga maritima (Tma UITma).

### B. Methods of Production

The fusion protein according to the present disclosure can be prepared recombinantly, by expression from e.g. a nucleic acid construct encoding for the fusion protein, for example as described in Molecular Cloning: A Laboratory Manual, 4^{th} edition (Sambrook et al., 2001), the entire contents of both of which are hereby incorporated by reference.

In one embodiment, DNA encoding the Csp and RT is isolated, respectively, and sequenced using conventional procedures (e.g., by using oligonucleotide probes that are capable of binding specifically to genes encoding the Csp or RT). The encoding DNA may also be obtained by synthetic methods. The isolated polynucleotide that encodes the Csp and RT can be inserted into a vector to generate a polynucleotide encoding the fusion protein using recombinant techniques known in the art. Many vectors are available. The vector components generally include, but are not limited to, one or more of the following: a signal sequence, an origin of replication, one or more marker genes, an enhancer element, a promoter (e.g. SV40, CMV, EF-1α), and a transcription termination sequence.

Vectors comprising the polynucleotide sequence encoding the fusion protein can be introduced to a host cell for cloning or gene expression. Suitable host cells for cloning or expressing the DNA in the vectors herein are the prokaryote (e.g., E. coli), yeast (e.g., Saccharomyces cerevisiae), or higher eukaryote cells (e.g., mammalian host cell lines).

Host cells are transfected with the above-described expression or cloning vectors for fusion protein production and cultured in conventional nutrient media modified as appropriate for inducing promoters, selecting transformants, or amplifying the genes encoding the desired sequences.

In certain embodiments, the fusion protein of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 10%, about 20%, about 30%, about 40%, about 50%, about 60%, about 70%, about 80%, about 90%, about 95% or more of the proteins (e.g., by weight) in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, or by heat denaturation, followed by centrifugation; gel filtration, reverse phase, hydroxylapatite and affinity chromatography; and combinations of such and other techniques.

### III. Compositions and Kits

Also provided in the present disclosure are compositions and kits comprising the fusion protein described herein. Such compositions and kits comprise, in addition to the fusion protein described herein, components usable for cDNA synthesis, such as primer, deoxyribonucleotide, and reaction buffer.

In one embodiment, the composition or kit according to the present disclosure may include at least one primer, at least one deoxyribonucleotide, and/or a reaction buffer solution in addition to the fusion protein described herein.

The primer may be an oligonucleotide having a nucleotide sequence complementary to the template RNA, and is not particularly limited as long as it anneals to the template RNA under the reaction conditions used. The primer may be oligonucleotide such as oligo (dT) or oligonucleotide having a random sequence (random primer).

The length of the primer is preferably at least six nucleotides since a specific annealing process is performed, and more preferably at least 10 nucleotides. The length of the primer is preferably at most 100 nucleotides and more preferably at most 30 nucleotides in terms of the synthesis of oligonucleotide. The oligonucleotide can be synthesized, for example, according to the phosphoramidite method by the DNA synthesizer 394 (manufactured by Applied Biosystems Inc). The oligonucleotide may be synthesized according to any other process, such as the triester phosphate method, H-phosphonate method, or thiophosphate method. The oligonucleotide may be oligonucleotide derived from a biological specimen, and for example, may be prepared such that it is isolated from restricted endonuclease digest of DNA prepared from a natural specimen.

As used herein, deoxyribonucleotide refers to phosphate groups bonded to deoxyribose bonded to organic bases by the phosphoester bond. A natural DNA includes four different nucleotides. The nucleotides respectively consisting of adenine, guanine, cytosine and thymine bases can be found in the natural DNA. The adenine, guanine, cytosine and thymine bases, are respectively abbreviated as A, G, C and T. The deoxyribonucleotide includes free monophosphate, diphosphate and triphosphate (more specifically, the phosphate groups each includes one, two or three phosphate portions). Therefore, the deoxyribonucleotide includes deoxyribonucleotide triphosphate (for example, dATP, dCTP, dITP, dGTP and dTTP) and derivatives thereof. The deoxyribonucleotide derivative includes [αS]dATP, 7-deaza-dGTP, 7-deaza-dATP and a deoxynucleotide derivative showing resistance against the decomposition of nucleic acid. The nucleotide derivative includes, for example, deoxyribonucleotide labeled in such a manner that can be detected by a radioactive isotope such as ³²P or ³⁵S, a fluorescent portion, a chemiluminescent portion, a bioluminescent portion or an enzyme.

Deoxyribonucleotide triphosphate, as used herein, refers to a nucleotide of which the sugar portion is composed of deoxyribose, and having a triphosphate group. A natural DNA includes four different nucleotides which respectively has adenine, guanine, cytosine and thymine as the base portion. The deoxyribonucleotide triphosphate contained in an exemplary composition or kit of the present disclosure is a mixture of four deoxyribonucleotides triphosphate, dATP, dCTP, dGTP, and dTTP.

As used herein, the reaction buffer solution means a solution suitable for the fusion protein disclosed herein to perform reverse transcription. In one embodiment, the reaction buffer includes a buffer agent or a buffer agent mixture and may further include divalent cations and monovalent cations. In one embodiment, the reaction buffer contained in the composition or kit is a 5× or 10× buffer solution, i.e., the buffer solution needs to be diluted 5 or 10 times in a reaction for reverse transcription. In one embodiment, the reaction buffer solution 1× contains 50 mM Tris 8.5, 50 mM KCl, and 4 mM MgCl₂.

### IV. Method of Use

In another aspect, the present disclosure provides methods of using the fusion protein as disclosed herein for cDNA synthesis.

In one embodiment, the method for synthesizing cDNA using the composition disclosed herein, comprises the steps of:
A) preparing a solution comprising the fusion protein disclosed herein, at least one primer, at least one deoxyribonucleotide, and RNA serving as a template; and
B) incubating the solution prepared in the step A) under a condition suitable for the fusion protein to perform reverse transcription reaction, i.e., synthesizing cDNA using the RNA as the template.

The RNA that can serve as a template for the method disclosed herein can be reverse transcribed reaction from a primer when the primer is hybridized to the RNA. In one method disclosed herein, the reverse transcription reaction may include one kind of template or a plurality of different templates having different nucleotide sequences. When a specific primer for a particular template is used, primer extension products from the plurality of different templates in the nucleic acid mixture can be produced. The plurality of templates may be present in the different nucleic acids or the same nucleic acid. The RNA, which is a template to which the method disclosed herein is applicable, is not particularly limited. Examples of the RNA are an group of RNA molecules in all of RNAs in a specimen, a group of RNA molecules such as mRNA, tRNA, and rRNA, or particular group of RNA molecules (for example, a group of RNA molecules having a common nucleotide sequence motif, a transcript by the RNA polymerase, a group of RNA molecules concentrated by means of the subtraction process), and an arbitrary RNA capable of producing the primer used in the reverse transcription reaction.

In some embodiments, the RNA serving as the template may be included in a specimen derived from an organism such as cells, tissues or blood, or a specimen such as food, soil or waste water which possibly includes organisms. Further, the RNA may be included in a nucleic acid-containing preparation obtained by processing such a specimen or the like according to the conventional process. Examples of the preparation is homogenized cells, and a specimen obtained by fractioning the homogenized cells, all of RNAs in the specimen, or a group of particular RNA molecules, for example, a specimen in which mRNA is enriched, and the like.

The amount of the fusion protein to be used in the method disclosed herein is not particularly limited. In the case where the reverse transcription reaction is performed with 20 μL of the reaction solution, the amount of the fusion protein can be 0.02-20 μg, or 1-10 μg, or 2-5 μg.

The concentration of the primer used in the method disclosed herein is not particularly limited. The concentration is preferably at least 0.1 μM in the reverse transcription reaction, preferably at least 2.5 μM in the case where an Oligo dT primer is used in the reverse transcription reaction, and preferably at least 5 μM in the case where a random primer is used in the reverse transcription reaction in order to maximize the cDNA synthesis from the template RNA.

The conditions which are suitable for the fusion protein to perform reverse transcription reaction, i.e., satisfactory for synthesizing the primer extension strand complementary to the template RNA are not particularly limited. An example of a temperature range may be 30° C.-65° C., preferably 37° C.-50° C., and 42° C.-45° C. is more preferable. An example of a preferable reaction time period is 5 min.-120 min., and 15 min.-60 min. is more preferable.

In certain embodiments, the cDNA synthesis method using the fusion protein disclosed herein has improved processivity compared to the reserve transcriptase from which the fusion protein is derived, when transcribing RNA template having a length of at least 5, 6, 7, 8, 9, 10, 11, 12, 13, 14, or 15 kb. As a result, the cDNA synthesis method disclosed herein is particularly advantageous in, for example, the synthesis of cDNA from long RNA template, i.e., a reverse transcription reaction requires high processivity. The improvement of the processivity of the reverse transcription reaction can evaluated by, for example, the examination of the amount and/or the strand length of the synthesized cDNA. The amount of synthesized cDNA obtained by the reverse transcription reaction can be examined such that a certain quantity of the reaction solution after the reverse transcription reaction is subjected to a real time PCR so that an amount of synthesized targeted nucleic acid sequence is quantified. The length of the synthesized cDNA obtained by the reverse transcription reaction can be confirmed by determining the amounts of the amplification products obtained in PCR using a pair of primer in different pairs of primers having different amplification strand lengths from the downstream vicinity of a priming region of the primer used in the reverse transcription reaction, and a certain quantity of the reaction solution after the reverse transcription reaction.

When a nucleic acid amplification reaction, wherein the cDNA obtained by the process according to the present invention is used as a template, is performed, the cDNA can be amplified. The nucleic acid amplification reaction is not particularly limited. A preferable example thereof is the polymerase chain reaction (PCR).

The following examples are provided to better illustrate the claimed invention and are not to be interpreted in any way as limiting the scope of the invention. All specific compositions, materials, and methods described below, in whole or in part, fall within the scope of the invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the invention. It is the intention of the inventors that such variations are included within the scope of the invention.

### Example 1

### Materials and Methods

Cloning of Various Fusion Constructs with MMLVmut

Unless stated otherwise, proteins were expressed with N-terminal His8 tag. Codon optimization for E. coli expression was carried out with IDT (Integrated DNA Technologies) software. The gene blocks were synthesized by Twist Bioscience and cloned into pET28a (Novagen) expression vector at NcoI and XhoI site. pET28a was first digested with NcoI (NEB, Cat #R3193) and XhoI (NEB, Cat #R0146) and linearized plasmid was gel purified using GFX PCR DNA and gel band purification kit (Cytiva, Cat #28903470). Expression plasmids assembly was done with HiFi DNA assembly master mix (New England Biolabs E2621) and sequence confirmed with Sanger sequencing (Azenta).

### Cloning of HIV2

ttCsp1 was fused at N-terminus of HIV p66 with a 10 amino acid linker. p51 has a C-terminal His8 tag. The gene fragment consisted of ttCsp1-p66-STOP-T7 promotor-rbs-p51-His8 was synthesized by Twist and Gibson assembled into pET28a vector at NcoI and XhoI site.

### Protein Expression and Purification

Plasmids were transformed into NiCo21 (DE3) cells (NEB, Cat #C2529H). Overnight culture was used to inoculate 400 ml of LB. Cells were grown at 37° C. until OD600 reaches 0.5-0.8, and induced with 0.2 mM IPTG at 16° C. for overnight. The next day, cells were pelleted and incubated in lysis buffer (30 mM Tris 8.0, 10 mM imidazole, 200 mM NaCl, 2 mM MgCl2, 10% glycerol, 0.5% n-octyl β-d-thioglucopyranoside, 10 mg of lysozyme for 20 minutes on ice. After adding DNase I, cell lysate was cleared for 1 h at 20000 g. 0.5 ml of Ni-NTA resin (Qiagen, Cat #30210) was added to the clarified lysate and batch bound for 1 h at 4° C. Ni-NTA resin was packed into an empty PD10 column (Cytiva, Cat #17043501) and washed with 100 ml of wash buffer (30 mM Tris 8.0, 30 mM imidazole, 300 mM NaCl, 5 mM MgSO₄, 10% glycerol, 0.5 mM DTT). Protein was eluted with 2.5 ml of elution buffer (30 mM Tris 8.0, 300 mM imidazole, 100 mM NaCl, 5 mM MgSO₄, 10% glycerol, and 1 mM DTT) and loaded onto a pre-equilibrated PD10 column (Cytiva, Cat #17085101) (equilibration buffer 50 mM Tris 8.0, 75 mM KCl, 3 mM MgCl₂, 10% glycerol, and 1 mM DTT), and eluted with 3.5 ml of the same equilibration buffer. The protein was then concentrated using Amicon Ultra-4 30K (Millipore, Cat #UFC803096) and glycerol was added to final 50% before it was aliquoted and snap frozen in liquid nitrogen and stored at −80° C. until future use. Before assays, protein concentrations were measured and equalized with Bradford assay (BioRad).

### Construction of Lambda and Hepatitis C RNA Ladders

pUC19 vector (NEB, Cat #N3041) was modified to include a T7 promoter, SpeI and PacI restriction digest sites, 30 bp from Lambda DNA followed by a stretch of A and a T7 terminator and NotI restriction digest site. The DNA fragment was synthesized by Twist and inserted into pUC19 at HindIII/XbaI sites. The DNA sequence between HindIII/XbaI is AAGCTTGAAATTAATACGACTCACTATAGGGGACTAGTTTAATTAAGTGATCCGA CAGGTTACGGGGTCCTGTCCGAAAAAAAAAGAAAAAAAAAAAACTAGCATAAC CCCTTGGGGCCTCTAAACGGGTCTTGAGGGGTTTTTTGGCGGCCGCTCTAGA (SEQ ID NO: 60). The final vector is pUC19T7A.

Different lengths of lambda DNA (NEB, Cat #N3011) was generated by PCR and assembled into pUC19T7A at SpeI site to obtain 1, 2, 3, 5, 7, and 9 kb ladder plasmids. The primers used for PCR are:

| | | SEQ |
|---|---|---|
| | | ID |
| Primer | Sequence (5′->3′) | No: |
| lambda | 5′-TGAAATTAATACGACTCACTATAGGGG | 61 |
| | ACTCCGCCACGACGATGAACAGAC | |
| 1R | 5′-CCGTAACCTGTCGGATCACTTAATTAA | 62 |
| | ACTGTCTGCGCGGGCATTGCCA | |
| 2R | 5′-CCGTAACCTGTCGGATCACTTAATTAA | 63 |
| | ACTCGCCCACGACTCGTTCGC | |
| 3R | 5′-CCGTAACCTGTCGGATCACTTAATTAA | 64 |
| | ACTGCGCAGCACCGTAATTACTGTG | |
| 5R | 5′-CCGTAACCTGTCGGATCACTTAATTAA | 65 |
| | ACTCGCTGTGCGTCCGCATCCT | |
| 7R | 5′-CCGTAACCTGTCGGATCACTTAATTAA | 66 |
| | ACTCTCCCCTTATAAACCCACAGGGT | |
| 9R | 5′-CCGTAACCTGTCGGATCACTTAATTAA | 67 |
| | ACTGCCTGATTGCCGGAAAGGACC | |

The 15 kb ladder plasmid was generated by digesting the 9 kb plasmid with PacI and insert a ˜6 kb lambda DNA piece generated with PCR using primers 5′-GGTCCTTTCCGGCAATCAGGCAGTTTAAT AGGGCTGACGTTTAACCAGAC (SEQ ID NO: 68) and 5′-CTGCGTGAGTATCCGTGAGAATAAGTGATCCGACAGGTTACGG (SEQ ID NO: 69).

To obtain the hepatitis C virus (HCV) RNA ladder, we first have to generate the HCV genome. HCV genome sequence NC_004102.1, serotype 1a, isolate H77 was used as the template for DNA fragment synthesis (Twist). The assembly into pUC19 vector was done with HiFi DNA assembly master mix (NEB E2621). Once HCV genome is constructed, the ladder plasmids were generated similarly to the lambda ladder plasmids. The primers used for PCR are:

| | | SEQ |
|---|---|---|
| | | ID |
| Primer | Sequence (5′->3′) | No: |
| T7AHCV_F | 5′-TGAAATTAATACGACTCACTATAGGGG | 70 |
| | ACTGCGACACTCCACCATGAATC | |
| HCV1R | 5′-CCGTAACCTGTCGGATCACTTAATTAA | 71 |
| | ACTAGGAATTGCGCACTTGGTAG | |
| HCV2R | 5′-CCGTAACCTGTCGGATCACTTAATTAA | 72 |
| | ACTGGTGGCCTGGTGTTGTTAAG | |
| HCV3R | 5′-CCGTAACCTGTCGGATCACTTAATTAA | 73 |
| | ACTCGAAGATGGCCAGGAGTAGT | |
| HCV5R | 5′-CCGTAACCTGTCGGATCACTTAATTAA | 74 |
| | ACTACGCCCTCCCAAAATTCAAG | |
| HCV7R | 5′-CCGTAACCTGTCGGATCACTTAATTAA | 75 |
| | ACTTCATCCTCCTCTGCCACAAG | |
| HCV9R | 5′-CCGTAACCTGTCGGATCACTTAATTAA | 76 |
| | ACTCGAGCAGGAGTAGGCAAAAC | |

Lambda ladder plasmids were linearized with NotI (NEB R3189) and HCV ladder plasmids were linearized with XbaI (NEB R0145). The plasmids were cleaned up using GFX PCR DNA and gel band purification kit (Cytiva 28903470) and RNA was transcribed using HiScribe T7 high yield RNA synthesis kit (NEB E2040S). RNA was purified with Monarch RNA cleanup kit (NEB T2050S). RNA concentrations were measured on a nanodrop machine and equal amounts of 2 μg/μl RNA of different sizes were combined to yield the final ladder.

### Reverse Transcriptase Assay

The 10 μl assay was set up as follows: 50 mM Tris 8.5, 50 mM KCl, 4 mM MgCl₂, 0.5 mM dNTP, 80 μM poly T primer (5′-TTTTTTTTTTTTCTTTTTTTTTCGG) (SEQ ID NO: 77), 1 μg of RNA ladder, 5 mM DTT with 1 μl of purified enzyme. The sample was incubated in PCR machine at different temperatures for various lengths of time. At the end of reaction, 2 μl 0.5 M EDTA and 2 μl 1 M NaOH were added and the mixture was incubated at 95° C. for 10′ to hydrolyze RNA. 4 μl 0.5 M HCl was used to neutralize the mixture. 2 μl of RNA input (assay without enzyme) and 2 μl of cDNA were mixed with 2×RNA loading dye (NEB B0363S) and analyzed on 0.8% agarose TAE gel.

### Example 2

This example illustrates that fusion of a cold shock protein (Csp) from Thermus thermophilus at the N-terminus of MMLV RT improves RT's processivity. Specifically, Csp1 from thermophilic microorganism Thermus thermophilus (ttCsp1) and the RNase H dead mutant of MMLV RT (D524N, E562Q, D583N, D653N) (MMLVmut) were used. Initially, the linker used was Gly-Ser-Gly-Gly-Ser (SEQ ID NO: 49, in fusion protein Bos6C1).

In order to test the processivity of RTs, we created lambda RNA ladder (1, 2, 3, 5, 7, 9, and 15 kb) and hepatitis C viral (HCV) RNA ladder (1, 2, 3, 5, 7, and 9 kb). All the ladders can be reverse transcribed with a common poly T primer.

HCV RNA is known to have complex folded structures (see Pirakitikulr et al., Mol Cell. 62 (1): 111-20 (2016); Quade et al., Nat Commun. 6:7646 (2015)) and would be a more difficult substrate for RTs. In particular, HCV RNA is a secondary structure-rich RNA, which is difficult to be reverse transcribed at lower temperatures (such as 37° C., 42° C. and so on). To reverse transcribe the HCV RNA, it is usually chosen to increase the temperature to uncoil the secondary structures. However, as the temperature increases, the RT that transcribes the HCV RNA must be more thermostable. In this example, we showed that our fusion protein can reverse transcribe HCV RNA at lower temperatures.

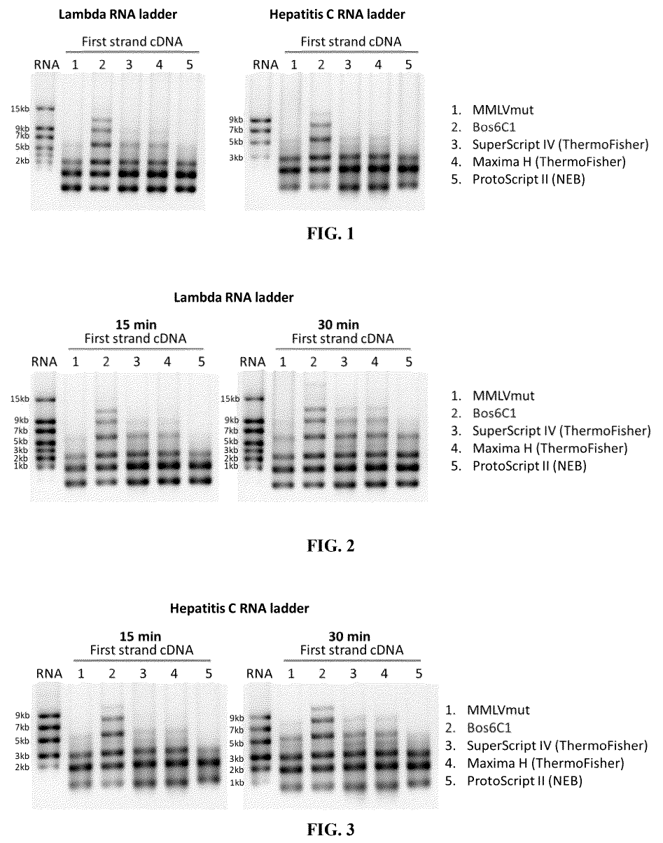

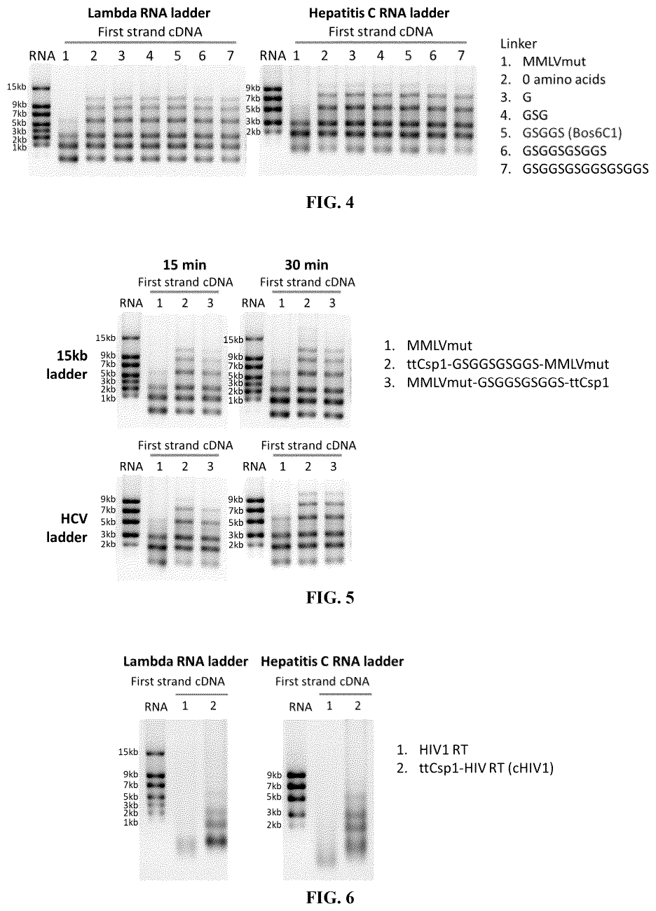

Comparison of Bos6C1 reverse transcription activity to commercial RTs (SuperScript IV, Maxima H (both from ThermoFisher) and ProtoScript II (NEB)) using both ladders showed Bos6C1 has much improved processivity (FIG. 1). Bos6C1 can reverse transcribe 9 kb RNA within 15′ at 42° C. After 30′ at 42° C., Bos6C1 can reach 15 kb, performs better than the other RTs (FIG. 2). In addition, Bos6C1 does not require reaction temperature increase to overcome the highly folded RNA structure in HCV RNA. Within 15 min at 42° C., Bos6C can complete first strand cDNA synthesis of the whole HCV genome, outperforms other RTs (FIG. 3).

### Example 3

This example illustrates the effect of the linker length on the RT function of the ttCsp1-MMLVmut fusion protein. To examine how the linker length affect the fusion protein function, the following constructs were made:

| | Poly- | | |
|---|---|---|---|
| | peptide | | |
| | SEQ ID | | Linker |
| Name | NO | Linker | SEQ ID NO |
| Bos6C4 | 78 | 0 amino acid | / |
| Bos6C5 | 79 | 1 amino acid | / |
| | | linker (G) | |
| Bos6C6 | 80 | 3 amino acids | / |
| | | linker (GSG) | |
| Bos6C1 | 81 | 5 amino acids | 49 |
| | | linker (GSGGS) | |
| Bos6C2 | 82 | 10 amino acids | 52 |
| | | linker | |
| | | (GSGGSGSGGS) | |
| Bos6C3 | 83 | 15 amino acids | 53 |
| | | linker | |
| | | (GSGGSGSGGSGSGGS) | |

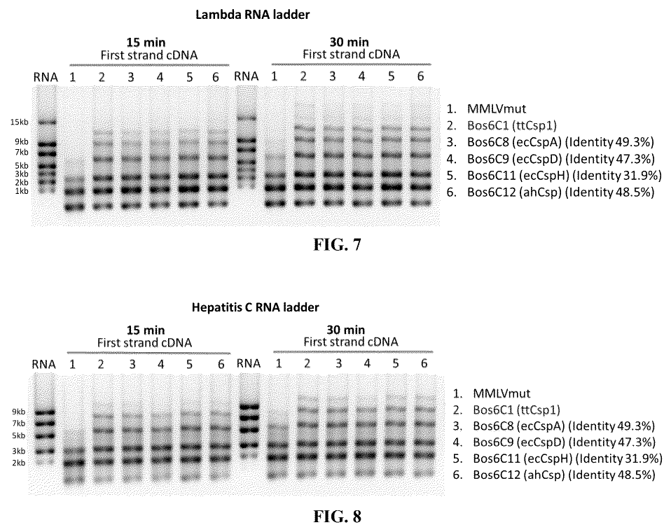

As shown in FIG. 4, linker length does not affect the fusion protein function. In addition, ttCsp1 can be fused at the C-terminus of MMLVmut (Bos6C7) to improve MMLVmut processivity (FIG. 5).

TABLE 4
Sequences of ttCsp1-MMLV Fusion Protein
With Different Linkers

| | | SEQ ID |
|---|---|---|
| Name | Sequence | NO: |
| Bos6C4 | MGSSHHHHHHHHGSGGSMQKGRVKWFNAEK | 78 |
| | GYGFIEREGDTDVFVHYTAINAKGFRTLNE | |
| | GDIVTFDVEPGRNGKGPQAVNVTVVEPARR | |
| | TLNIEDEYRLHETSKEPDVSLGSTWLSDFP | |
| | QAWAETGGMGLAVRQAPLIIPLKATSTPVS | |
| | IKQYPMSQEARLGIKPHIQRLLDQGILVPC | |
| | QSPWNTPLLPVKKPGTNDYRPVQDLREVNK | |
| | RVEDIHPTVPNPYNLLSGLPPSHQWYTVLD | |
| | LKDAFFCLRLHPTSQPLFAFEWRDPEMGIS | |
| | GQLTWTRLPQGFKNSPTLFDEALHRDLADF | |
| | RIQHPDLILLQYVDDLLLAATSELDCQQGT | |
| | RALLQTLGNLGYRASAKKAQICQKQVKYLG | |
| | YLLKEGQRWLTEARKETVMGQPTPKTPRQL | |
| | REFLGTAGFCRLWIPGFAEMAAPLYPLTKT | |
| | GTLFNWGPDQQKAYQEIKQALLTAPALGLP | |
| | DLTKPFELFVDEKQGYAKGVLTQKLGPWRR | |
| | PVAYLSKKLDPVAAGWPPCLRMVAAIAVLT | |
| | KDAGKLTMGQPLVILAPHAVEALVKQPPDR | |
| | WLSNARMTHYQALLLDTDRVQFGPVVALNP | |
| | ATLLPLPEEGLQHNCLDILAEAHGTRPDLT | |
| | DQPLPDADHTWYTNGSSLLQEGQRKAGAAV | |
| | TTETEVIWAKALPAGTSAQRAQLIALTQAL | |
| | KMAEGKKLNVYTNSRYAFATAHIHGEIYRR | |
| | RGLLTSEGKEIKNKDEILALLKALFLPKRL | |
| | SIIHCPGHQKGHSAEARGNRMANQAARKAA | |
| | ITETPDTSTLL | |
| Bos6C5 | MGSSHHHHHHHHGSGGSMQKGRVKWFNAEK | 79 |
| | GYGFIEREGDTDVFVHYTAINAKGFRTLNE | |
| | GDIVTFDVEPGRNGKGPQAVNVTVVEPARR | |
| | GTLNIEDEYRLHETSKEPDVSLGSTWLSDF | |
| | PQAWAETGGMGLAVRQAPLIIPLKATSTPV | |
| | SIKQYPMSQEARLGIKPHIQRLLDQGILVP | |
| | CQSPWNTPLLPVKKPGTNDYRPVQDLREVN | |
| | KRVEDIHPTVPNPYNLLSGLPPSHQWYTVL | |
| | DLKDAFFCLRLHPTSQPLFAFEWRDPEMGI | |
| | SGQLTWTRLPQGFKNSPTLFDEALHRDLAD | |
| | FRIQHPDLILLQYVDDLLLAATSELDCQQG | |
| | TRALLQTLGNLGYRASAKKAQICQKQVKYL | |
| | GYLLKEGQRWLTEARKETVMGQPTPKTPRQ | |
| | LREFLGTAGFCRLWIPGFAEMAAPLYPLTK | |
| | TGTLFNWGPDQQKAYQEIKQALLTAPALGL | |
| | PDLTKPFELFVDEKQGYAKGVLTQKLGPWR | |
| | RPVAYLSKKLDPVAAGWPPCLRMVAAIAVL | |
| | TKDAGKLTMGQPLVILAPHAVEALVKQPPD | |
| | RWLSNARMTHYQALLLDTDRVQFGPVVALN | |
| | PATLLPLPEEGLQHNCLDILAEAHGTRPDL | |
| | TDQPLPDADHTWYTNGSSLLQEGQRKAGAA | |
| | VTTETEVIWAKALPAGTSAQRAQLIALTQA | |
| | LKMAEGKKLNVYTNSRYAFATAHIHGEIYR | |
| | RRGLLTSEGKEIKNKDEILALLKALFLPKR | |
| | LSIIHCPGHQKGHSAEARGNRMANQAARKA | |
| | AITETPDTSTLL | |
| Bos6C6 | MGSSHHHHHHHHGSGGSMQKGRVKWFNAEK | 80 |
| | GYGFIEREGDTDVFVHYTAINAKGFRTLNE | |
| | GDIVTFDVEPGRNGKGPQAVNVTVVEPARR | |
| | GSGTLNIEDEYRLHETSKEPDVSLGSTWLS | |
| | DFPQAWAETGGMGLAVRQAPLIIPLKATST | |
| | PVSIKQYPMSQEARLGIKPHIQRLLDQGIL | |
| | VPCQSPWNTPLLPVKKPGTNDYRPVQDLRE | |
| | VNKRVEDIHPTVPNPYNLLSGLPPSHQWYT | |
| | VLDLKDAFFCLRLHPTSQPLFAFEWRDPEM | |
| | GISGQLTWTRLPQGFKNSPTLFDEALHRDL | |
| | ADFRIQHPDLILLQYVDDLLLAATSELDCQ | |
| | QGTRALLQTLGNLGYRASAKKAQICQKQVK | |
| | YLGYLLKEGQRWLTEARKETVMGQPTPKTP | |
| | RQLREFLGTAGFCRLWIPGFAEMAAPLYPL | |
| | TKTGTLFNWGPDQQKAYQEIKQALLTAPAL | |
| | GLPDLTKPFELFVDEKQGYAKGVLTQKLGP | |
| | WRRPVAYLSKKLDPVAAGWPPCLRMVAAIA | |
| | VLTKDAGKLTMGQPLVILAPHAVEALVKQP | |
| | PDRWLSNARMTHYQALLLDTDRVQFGPVVA | |
| | LNPATLLPLPEEGLQHNCLDILAEAHGTRP | |
| | DLTDQPLPDADHTWYTNGSSLLQEGQRKAG | |
| | AAVTTETEVIWAKALPAGTSAQRAQLIALT | |
| | QALKMAEGKKLNVYTNSRYAFATAHIHGEI | |
| | YRRRGLLTSEGKEIKNKDEILALLKALFLP | |
| | KRLSIIHCPGHQKGHSAEARGNRMANQAAR | |
| | KAAITETPDTSTLL | |
| Bos6C1 | MGSSHHHHHHHHGSGGSMQKGRVKWFNAEK | 81 |
| | GYGFIEREGDTDVFVHYTAINAKGFRTLNE | |
| | GDIVTFDVEPGRNGKGPQAVNVTVVEPARR | |
| | GSGGSTLNIEDEYRLHETSKEPDVSLGSTW | |
| | LSDFPQAWAETGGMGLAVRQAPLIIPLKAT | |
| | STPVSIKQYPMSQEARLGIKPHIQRLLDQG | |
| | ILVPCQSPWNTPLLPVKKPGTNDYRPVQDL | |
| | REVNKRVEDIHPTVPNPYNLLSGLPPSHQW | |
| | YTVLDLKDAFFCLRLHPTSQPLFAFEWRDP | |
| | EMGISGQLTWTRLPQGFKNSPTLFDEALHR | |
| | DLADFRIQHPDLILLQYVDDLLLAATSELD | |
| | CQQGTRALLQTLGNLGYRASAKKAQICQKQ | |
| | VKYLGYLLKEGQRWLTEARKETVMGQPTPK | |
| | TPRQLREFLGTAGFCRLWIPGFAEMAAPLY | |
| | PLTKTGTLFNWGPDQQKAYQEIKQALLTAP | |
| | ALGLPDLTKPFELFVDEKQGYAKGVLTQKL | |
| | GPWRRPVAYLSKKLDPVAAGWPPCLRMVAA | |
| | IAVLTKDAGKLTMGQPLVILAPHAVEALVK | |
| | QPPDRWLSNARMTHYQALLLDTDRVQFGPV | |
| | VALNPATLLPLPEEGLQHNCLDILAEAHGT | |
| | RPDLTDQPLPDADHTWYTNGSSLLQEGQRK | |
| | AGAAVTTETEVIWAKALPAGTSAQRAQLIA | |
| | LTQALKMAEGKKLNVYTNSRYAFATAHIHG | |
| | EIYRRRGLLTSEGKEIKNKDEILALLKALF | |
| | LPKRLSIIHCPGHQKGHSAEARGNRMANQA | |
| | ARKAAITETPDTSTLL | |
| Bos6C2 | MGSSHHHHHHHHGSGGSMQKGRVKWFNAEK | 82 |
| | GYGFIEREGDTDVFVHYTAINAKGFRTLNE | |
| | GDIVTFDVEPGRNGKGPQAVNVTVVEPARR | |
| | GSGGSGSGGSTLNIEDEYRLHETSKEPDVS | |
| | LGSTWLSDFPQAWAETGGMGLAVRQAPLII | |
| | PLKATSTPVSIKQYPMSQEARLGIKPHIQR | |
| | LLDQGILVPCQSPWNTPLLPVKKPGTNDYR | |
| | PVQDLREVNKRVEDIHPTVPNPYNLLSGLP | |
| | PSHQWYTVLDLKDAFFCLRLHPTSQPLFAF | |
| | EWRDPEMGISGQLTWTRLPQGFKNSPTLFD | |
| | EALHRDLADFRIQHPDLILLQYVDDLLLAA | |
| | TSELDCQQGTRALLQTLGNLGYRASAKKAQ | |
| | ICQKQVKYLGYLLKEGQRWLTEARKETVMG | |
| | QPTPKTPRQLREFLGTAGFCRLWIPGFAEM | |
| | AAPLYPLTKTGTLFNWGPDQQKAYQEIKQA | |
| | LLTAPALGLPDLTKPFELFVDEKQGYAKGV | |
| | LTQKLGPWRRPVAYLSKKLDPVAAGWPPCL | |
| | RMVAAIAVLTKDAGKLTMGQPLVILAPHAV | |
| | EALVKQPPDRWLSNARMTHYQALLLDTDR | |
| | VQFGPVVALNPATLLPLPEEGLQHNCLDIL | |
| | AEAHGTRPDLTDQPLPDADHTWYTNGSSLL | |
| | QEGQRKAGAAVTTETEVIWAKALPAGTSAQ | |
| | RAQLIALTQALKMAEGKKLNVYTNSRYAFA | |
| | TAHIHGEIYRRRGLLTSEGKEIKNKDEILA | |
| | LLKALFLPKRLSIIHCPGHQKGHSAEARGN | |
| | RMANQAARKAAITETPDTSTLL | |
| Bos6C3 | MGSSHHHHHHHHGSGGSMQKGRVKWFNAEK | 83 |
| | GYGFIEREGDTDVFVHYTAINAKGFRTLNE | |
| | GDIVTFDVEPGRNGKGPQAVNVTVVEPARR | |
| | GSGGSGSGGSGSGGSTLNIEDEYRLHETSK | |
| | EPDVSLGSTWLSDFPQAWAETGGMGLAVRQ | |
| | APLIIPLKATSTPVSIKQYPMSQEARLGIK | |
| | PHIQRLLDQGILVPCQSPWNTPLLPVKKPG | |
| | TNDYRPVQDLREVNKRVEDIHPTVPNPYNL | |
| | LSGLPPSHQWYTVLDLKDAFFCLRLHPTSQ | |
| | PLFAFEWRDPEMGISGQLTWTRLPQGFKNS | |
| | PTLFDEALHRDLADFRIQHPDLILLQYVDD | |
| | LLLAATSELDCQQGTRALLQTLGNLGYRAS | |
| | AKKAQICQKQVKYLGYLLKEGQRWLTEARK | |
| | ETVMGQPTPKTPRQLREFLGTAGFCRLWIP | |
| | GFAEMAAPLYPLTKTGTLFNWGPDQQKAYQ | |
| | EIKQALLTAPALGLPDLTKPFELFVDEKQG | |
| | YAKGVLTQKLGPWRRPVAYLSKKLDPVAAG | |
| | WPPCLRMVAAIAVLTKDAGKLTMGQPLVIL | |
| | APHAVEALVKQPPDRWLSNARMTHYQALLL | |
| | DTDRVQFGPVVALNPATLLPLPEEGLQHNC | |
| | LDILAEAHGTRPDLTDQPLPDADHTWYTNG | |
| | SSLLQEGQRKAGAAVTTETEVIWAKALPAG | |
| | TSAQRAQLIALTQALKMAEGKKLNVYTNSR | |
| | YAFATAHIHGEIYRRRGLLTSEGKEIKNKD | |
| | EILALLKALFLPKRLSIIHCPGHQKGHSAE | |
| | ARGNRMANQAARKAAITETPDTSTLL | |

### Example 4

This example illustrates that ttCsp1 can also improve the processivity of HIV1 RT. To test if ttCsp1 can help other RTs' processivity, HIV1 RT was picked since it is a more thoroughly studied viral RT. HIV1 RT has two subunits, p66 and p51. p51 is a proteolytic cleavage product of p66. Together, they form a tight complex and both parts are needed for HIV1 RT stability and function. Looking at the structure of HIV1 RT (PDB 4B3O), the N-terminus of p66 is closer to the active site. Therefore, ttCsp1 was fused to the N-terminus of HIV1 p66 subunit (cHIV1). As shown in FIG. 6, cHIV1 can reverse transcribe much longer RNA pieces than HIV1 RT itself, demonstrating ttCsp1 fusion can be used as a general strategy to improve processivity of RTs.

TABLE 5
Sequences of HIV1 RT Subnits

| Name | Sequence | NO: |
|---|---|---|
| cHIV p66 | MGSQKGRVKWFNAEKGYGFIEREGDTDVFV | 84 |
| | HYTAINAKGFRTLNEGDIVTFDVEPGRNGK | |
| | GPQAVNVTVVEPARRGSGGSGSGGSPISPI | |
| | ETVPVKLKPGMDGPKVKQWPLTEEKIKALV | |
| | EICTEMEKEGKISKIGPENPYNTPVFAIKK | |
| | KDGTKWRKLVDFRELNKKTQDFWEVQLGIP | |
| | HPAGLKKKKSVTVLDVGDAYFSVPLDEDFR | |
| | KYTAFTIPSINNETPGIRYQYNVLPQGWKG | |
| | SPAIFQSSMTKILEPFRKQNPDIVIYQYMD | |
| | DLYVGSDLEIGQHRTKIEELRQHLLRWGLT | |
| | TPDKKHQKEPPFLWMGYELHPDKWTVQPIV | |
| | LPEKDSWTVNDIQKLVGKLNWASQIYPGIK | |
| | VRQLSKLLRGTKALTEVIPLTEEAELELA | |
| | ENREILKEPVHGVYYDPSKDLIAEIQKQGQ | |
| | GQWTYQIYQEPFKNLKTGKYARMRGAHTND | |
| | VKQLTEAVQKITTESIVIWGKTPKFKLPIQ | |
| | KETWETWWTEYWQATWVPEWEFVNTPPLVK | |
| | LWYQLEKEPIVGAETFYVNGAASRETKLGK | |
| | AGYVTNKGRQKVVTLTDTTNQKTQLQAIHL | |
| | ALQDSGLEVNIVTNSQYALGIIQAQPDQSE | |
| | SELVNQIIEQLIKKEKVYLAWVPAHKGIGG | |
| | NEQVDKLVSAGIRKIL | |
| cHIC p51 | MGPISPIETVPVKLKPGMDGPKVKQWPLTE | 85 |
| | EKIKALVEICTEMEKEGKISKIGPENPYNT | |
| | PVFAIKKKDGTKWRKLVDFRELNKKTQDFW | |
| | EVQLGIPHPAGLKKKKSVTVLDVGDAYFSV | |
| | PLDEDFRKYTAFTIPSINNETPGIRYQYNV | |
| | LPQGWKGSPAIFQSSMTKILEPFRKQNPDI | |
| | VIYQYMDDLYVGSDLEIGQHRTKIEELRQH | |
| | LLRWGLTTPDKKHQKEPPFLWMGYELHPDK | |
| | WTVQPIVLPEKDSWTVNDIQKLVGKLNWAS | |
| | QIYPGIKVRQLSKLLRGTKALTEVIPLTEE | |
| | AELELAENREILKEPVHGVYYDPSKDLIAE | |
| | IQKQGQGQWTYQIYQEPFKNLKTGKYARMR | |
| | GAHTNDVKQLTEAVQKITTESIVIWGKTPK | |
| | FKLPIQKETWETWWTEYWQATWVPEWEFVN | |
| | TPPLVKLWYQGSGGSSHHHHHHHH | |

### Example 5

This example illustrates that Csps other than ttCsp1 can also improve RT's processivity.

Cold shock proteins are highly abundant (see Yu et al., Int J Mol Sci. 20 (16): 4059 (2019)). E. coli has 9 genes (CspA to CspI) encoding proteins with similar fold and function. ecCspA has been demonstrated to be an RNA chaperon (see Jiang et al., J Biol Chem. 272 (1): 196-202 (1997)) while the functions of other ecCsp proteins are less clear. The crystal structure of Bacillus subtilis CspB with DNA dT6 (see Max et al., J Mol Biol. 360 (3): 702-14 (2006)) showed the important interactions between Csp and nucleic acids is base-stacking-to-hydrophobic residues without significant interaction with the backbone of the nucleic acids. Therefore, we hypothesized that all Csp proteins with similar fold and correctly positioned hydrophobic residues can be fused to MMLVmut to improve its processivity, regardless of Csp's in vivo biological function or the nature of its interacting nucleic acids. To demonstrate this, a series of different Csp to MMLVmut fusions were constructed, all at the N-terminus of MMLVmut with 5 amino acids (GSGGS, SEQ ID NO: 41) linker. (ec=E. coli, ah=Actinomadura harenae)

TABLE 6
MMLV Fused with Csps from
E. Coli or A. harenae

| | Poly- | | |
|---|---|---|---|
| | peptide | | Similarity/ |
| | SEQ ID | | identity to |
| Name | NO | Description | ttCsp1 |
| Bos6C8 | 86 | ecCspA-MMLVmut | 78.1%/49.3% |
| Bos6C9 | 87 | ecCspD-MMLVmut | 70.8%/47.3% |
| Bos6C11 | 88 | ecCspH-MMLVmut | 57.1%/31.9% |
| Bos6C12 | 89 | ahCsp-MMLVmut | 49%/48.5% |

TABLE 7
Sequences of MMLV Fused with Csps
from E. coli or A. harenae

| | Sequence | SEQ ID |
|---|---|---|
| Name | | NO: |
| Bos6C8 | MGSSHHHHHHHHGSGGSMSGKMTGIVKWFN | 86 |
| | ADKGFGFITPDDGSKDVFVHFSAIQNDGYK | |
| | SLDEGQKVSFTIESGAKGPAAGNVTSLGSG | |
| | GSTLNIEDEYRLHETSKEPDVSLGSTWLSD | |
| | FPQAWAETGGMGLAVRQAPLIIPLKATSTP | |
| | VSIKQYPMSQEARLGIKPHIQRLLDQGILV | |
| | PCQSPWNTPLLPVKKPGTNDYRPVQDLREV | |
| | NKRVEDIHPTVPNPYNLLSGLPPSHQWYTV | |
| | LDLKDAFFCLRLHPTSQPLFAFEWRDPEMG | |
| | ISGQLTWTRLPQGFKNSPTLFDEALHRDLA | |
| | DFRIQHPDLILLQYVDDLLLAATSELDCQQ | |
| | GTRALLQTLGNLGYRASAKKAQICQKQVKY | |
| | LGYLLKEGQRWLTEARKETVMGQPTPKTPR | |
| | QLREFLGTAGFCRLWIPGFAEMAAPLYPLT | |
| | KTGTLFNWGPDQQKAYQEIKQALLTAPALG | |
| | LPDLTKPFELFVDEKQGYAKGVLTQKLGPW | |
| | RRPVAYLSKKLDPVAAGWPPCLRMVAAIAV | |
| | LTKDAGKLTMGQPLVILAPHAVEALVKQPP | |
| | DRWLSNARMTHYQALLLDTDRVQFGPVVAL | |
| | NPATLLPLPEEGLQHNCLDILAEAHGTRPD | |
| | LTDQPLPDADHTWYTNGSSLLQEGQRKAGA | |
| | AVTTETEVIWAKALPAGTSAQRAQLIALTQ | |
| | ALKMAEGKKLNVYTNSRYAFATAHIHGEIY | |
| | RRRGLLTSEGKEIKNKDEILALLKALFLPK | |
| | RLSIIHCPGHQKGHSAEARGNRMANQAARK | |
| | AAITETPDTSTLL | |
| Bos6C9 | MGSSHHHHHHHHGSGGSMEKGTVKWFNNAK | 87 |
| | GFGFICPEGGGEDIFAHYSTIQMDGYRTLK | |
| | AGQSVQFDVHQGPKGNHASVIVPVEVEAAV | |
| | AGSGGSTLNIEDEYRLHETSKEPDVSLGST | |
| | WLSDFPQAWAETGGMGLAVRQAPLIIPLKA | |
| | TSTPVSIKQYPMSQEARLGIKPHIQRLLDQ | |
| | GILVPCQSPWNTPLLPVKKPGTNDYRPVQD | |
| | LREVNKRVEDIHPTVPNPYNLLSGLPPSHQ | |
| | WYTVLDLKDAFFCLRLHPTSQPLFAFEWRD | |
| | PEMGISGQLTWTRLPQGFKNSPTLFDEALH | |
| | RDLADFRIQHPDLILLQYVDDLLLAATSEL | |
| | DCQQGTRALLQTLGNLGYRASAKKAQICQK | |
| | QVKYLGYLLKEGQRWLTEARKETVMGQPTP | |
| | KTPRQLREFLGTAGFCRLWIPGFAEMAAPL | |
| | YPLTKTGTLFNWGPDQQKAYQEIKQALLTA | |
| | PALGLPDLTKPFELFVDEKQGYAKGVLTQK | |
| | LGPWRRPVAYLSKKLDPVAAGWPPCLRMVA | |
| | AIAVLTKDAGKLTMGQPLVILAPHAVEALV | |
| | KQPPDRWLSNARMTHYQALLLDTDRVQFGP | |
| | VVALNPATLLPLPEEGLQHNCLDILAEAHG | |
| | TRPDLTDQPLPDADHTWYTNGSSLLQEGQR | |
| | KAGAAVTTETEVIWAKALPAGTSAQRAQLI | |
| | ALTQALKMAEGKKLNVYTNSRYAFATAHIH | |
| | GEIYRRRGLLTSEGKEIKNKDEILALLKAL | |
| | FLPKRLSIIHCPGHQKGHSAEARGNRMANQ | |
| | AARKAAITETPDTSTLL | |
| Bos6C11 | MGSSHHHHHHHHGSGGSMSRKMTGIVKTFD | 88 |
| | RKSGKGFIIPSDGRKEVQVHISAFTPRDAE | |
| | VLIPGLRVEFCRVNGLRGPTAANVYLSGSG | |
| | GSTLNIEDEYRLHETSKEPDVSLGSTWLSD | |
| | FPQAWAETGGMGLAVRQAPLIIPLKATSTP | |
| | VSIKQYPMSQEARLGIKPHIQRLLDQGILV | |
| | PCQSPWNTPLLPVKKPGTNDYRPVQDLREV | |
| | NKRVEDIHPTVPNPYNLLSGLPPSHQWYTV | |
| | LDLKDAFFCLRLHPTSQPLFAFEWRDPEMG | |
| | ISGQLTWTRLPQGFKNSPTLFDEALHRDLA | |
| | DFRIQHPDLILLQYVDDLLLAATSELDCQQ | |
| | GTRALLQTLGNLGYRASAKKAQICQKQVKY | |
| | LGYLLKEGQRWLTEARKETVMGQPTPKTPR | |
| | QLREFLGTAGFCRLWIPGFAEMAAPLYPLT | |
| | KTGTLFNWGPDQQKAYQEIKQALLTAPALG | |
| | LPDLTKPFELFVDEKQGYAKGVLTQKLGPW | |
| | RRPVAYLSKKLDPVAAGWPPCLRMVAAIAV | |
| | LTKDAGKLTMGQPLVILAPHAVEALVKQPP | |
| | DRWLSNARMTHYQALLLDTDRVQFGPVVAL | |
| | NPATLLPLPEEGLQHNCLDILAEAHGTRPD | |
| | LTDQPLPDADHTWYTNGSSLLQEGQRKAGA | |
| | AVTTETEVIWAKALPAGTSAQRAQLIALTQ | |
| | ALKMAEGKKLNVYTNSRYAFATAHIHGEIY | |
| | RRRGLLTSEGKEIKNKDEILALLKALFLPK | |
| | RLSIIHCPGHQKGHSAEARGNRMANQAARK | |
| | AAITETPDTSTLL | |
| Bos6C12 | MGSSHHHHHHHHGSGGSMAQGTVKWFNADK | 89 |
| | GYGFIAVDGGADVFVHYSVIQMDGYRSLEQ | |
| | GQRVEFEITQSDRGPQAESVRLLGSGGSGS | |
| | GGSTLNIEDEYRLHETSKEPDVSLGSTWLS | |
| | DFPQAWAETGGMGLAVRQAPLIIPLKATST | |
| | PVSIKQYPMSQEARLGIKPHIQRLLDQGIL | |
| | VPCQSPWNTPLLPVKKPGTNDYRPVQDLRE | |
| | VNKRVEDIHPTVPNPYNLLSGLPPSHQWYT | |
| | VLDLKDAFFCLRLHPTSQPLFAFEWRDPEM | |
| | GISGQLTWTRLPQGFKNSPTLFDEALHRDL | |
| | ADFRIQHPDLILLQYVDDLLLAATSELDCQ | |
| | QGTRALLQTLGNLGYRASAKKAQICQKQVK | |
| | YLGYLLKEGQRWLTEARKETVMGQPTPKTP | |
| | RQLREFLGTAGFCRLWIPGFAEMAAPLYPL | |
| | TKTGTLFNWGPDQQKAYQEIKQALLTAPAL | |
| | GLPDLTKPFELFVDEKQGYAKGVLTQKLGP | |
| | WRRPVAYLSKKLDPVAAGWPPCLRMVAAIA | |
| | VLTKDAGKLTMGQPLVILAPHAVEALVKQP | |
| | PDRWLSNARMTHYQALLLDTDRVQFGPVVA | |
| | LNPATLLPLPEEGLQHNCLDILAEAHGTRP | |
| | DLTDQPLPDADHTWYTNGSSLLQEGQRKAG | |
| | AAVTTETEVIWAKALPAGTSAQRAQLIALT | |
| | QALKMAEGKKLNVYTNSRYAFATAHIHGEI | |
| | YRRRGLLTSEGKEIKNKDEILALLKALFLP | |
| | KRLSIIHCPGHQKGHSAEARGNRMANQAAR | |
| | KAAITETPDTSTLL | |

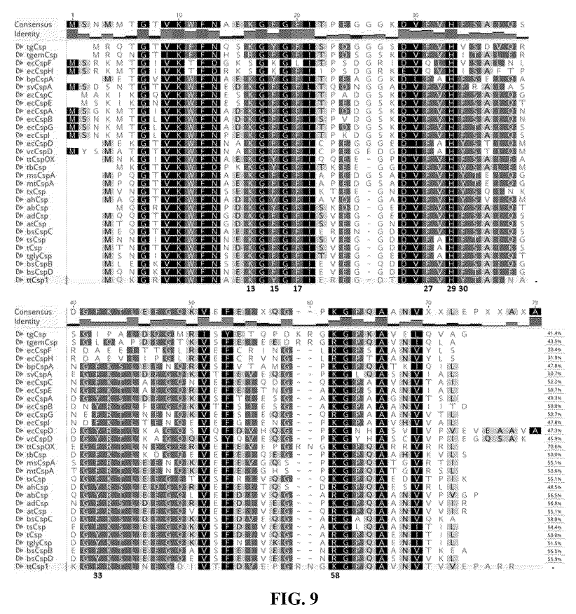

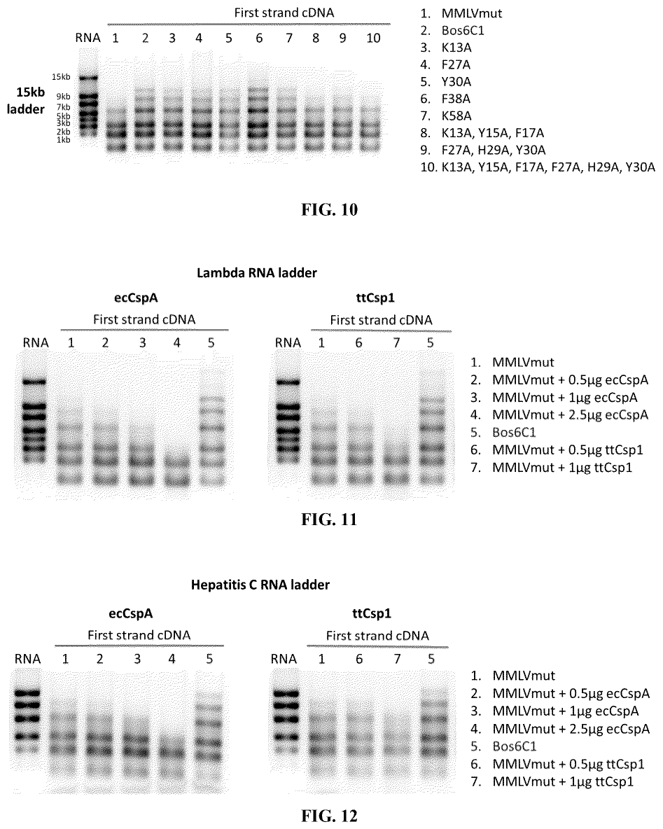

All fusion proteins showed improvement in processivity, similar to Bos6C1 (FIG. 7 and FIG. 8). In addition, various residues important for nucleic acids interaction were identified in the bsCspB structure (see Max et al., J Mol Biol. 360 (3): 702-14). Based on sequence alignment (FIG. 9), mutation studies were conducted on Bos6C1 to demonstrate the importance of these residues for improving fusion RT processivity in ttCsp1 background (FIG. 10). The result shows single or multiple mutations of important and conserved nucleic acid binding residues decrease fusion RT processivity, but do not completely abolish the improvement over MMLVmut alone, indicating multiple residues contribute to nucleic acids binding. There is a general correlation between the mutations that increased K_{D} of bsCspB to dT6 (Max et al., J Mol Biol. 360 (3): 702-14, Table 4) and the mutations that impaired fusion RT's ability to transcribe longer pieces of RNA, indicating the similar mechanisms of RNA interaction utilized by ttCsp1 in the fusion RT protein. However, residual improvement in processivity of Csp mutants compared to MMLVmut indicates that the small Csp protein fold utilizes multiple residues for nucleic acids binding.

### Example 6

As Csp has been previously reported to be able to increase RT activity when combining Csp and RT in a reverse transcription reaction (see, WO2009/108949A2), we compared the processivity of the fusion protein disclosed herein and the combination of free Csp (ecCspA and ttCsp1 respectively) and RT. As shown in FIG. 11 and FIG. 12, while the fusion protein improved processivity of reverse transcription from a long RNA template, free Csp actually hindered reverse transcription of longer RNA pieces.

In conclusion, Csp protein fold uniquely recognizes nucleic acid segments in a sequence and backbone independent manner via protein hydrophobic residue to nucleic acid base stacking interaction. This property can be explored to help localize reverse transcriptase to RNA, improve their association with RNA as well as unfold RNA structure, resulting in overall improvement in RT processivity.

### Example 7

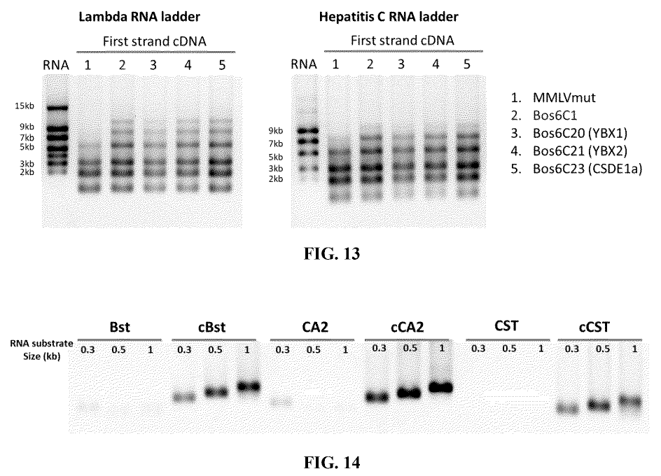

This example illustrates that Csp-like domains in eukaryotic proteins can improve RT activity. We fused three Csp-like domains from human proteins, YBX1, YBX2, and CSDE1a, with MMLV RT separately. As shown in FIG. 13, Csp-like domains in eukaryotic proteins can also improve RT activity.

Notably, YBX1 or YBX2 have the structural motif (SEQ ID NO: 1), RNP1 motif (SEQ ID NO: 2) and RNP2 motif (SEQ ID NO: 3); CSDE1a has RNP1 motif (SEQ ID NO: 2). The results shown in FIGS. 10 and 13 demonstrate that RNP1 and RNP2 play an important role in enhancing processivity of reverse transcriptase. Csp-type fold and its nucleic acid interacting residues are keys to improve RT processivity, regardless of domain/protein origin.

TABLE 7
MMLVmut Fused with Eukaryotic Csp-like Domain

| | | SEQ ID |
|---|---|---|
| Name | Sequence | NO: |
| Bos6C20 | MGSSHHHHHHHHGSGGSDKKVIATKVLGTV | 90 |
| | KWFNVRNGYGFINRNDTKEDVFVHQTAIKK | |
| | NNPRKYLRSVGDGETVEFDVVEGEKGAEAA | |
| | NVTGPGSGGSTLNIEDEYRLHETSKEPDVS | |
| | LGSTWLSDFPQAWAETGGMGLAVRQAPLII | |
| | PLKATSTPVSIKQYPMSQEARLGIKPHIQR | |
| | LLDQGILVPCQSPWNTPLLPVKKPGTNDYR | |
| | PVQDLREVNKRVEDIHPTVPNPYNLLSGLP | |
| | PSHQWYTVLDLKDAFFCLRLHPTSQPLFAF | |
| | EWRDPEMGISGQLTWTRLPQGFKNSPTLFD | |
| | EALHRDLADFRIQHPDLILLQYVDDLLLAA | |
| | TSELDCQQGTRALLQTLGNLGYRASAKKAQ | |
| | ICQKQVKYLGYLLKEGQRWLTEARKETVMG | |
| | QPTPKTPRQLREFLGTAGFCRLWIPGFAEM | |
| | AAPLYPLTKTGTLFNWGPDQQKAYQEIKQA | |
| | LLTAPALGLPDLTKPFELFVDEKQGYAKGV | |
| | LTQKLGPWRRPVAYLSKKLDPVAAGWPPCL | |
| | RMVAAIAVLTKDAGKLTMGQPLVILAPHAV | |
| | EALVKQPPDRWLSNARMTHYQALLLDTDRV | |
| | QFGPVVALNPATLLPLPEEGLQHNCLDILA | |
| | EAHGTRPDLTDQPLPDADHTWYTNGSSLLQ | |
| | EGQRKAGAAVTTETEVIWAKALPAGTSAQR | |
| | AQLIALTQALKMAEGKKLNVYTNSRYAFAT | |
| | AHIHGEIYRRRGLLTSEGKEIKNKDEILAL | |
| | LKALFLPKRLSIIHCPGHQKGHSAEARGNR | |
| | MANQAARKAAITETPDTSTLL | |
| Bos6C21 | MGSSHHHHHHHHGSGGSADKPVLATKVLGT | 91 |
| | VKWFNVRNGYGFINRNDTKEDVFVHQTAIK | |
| | RNNPRKFLRSVGDGETVEFDVVEGEKGAEA | |
| | TNVTGPGGVPVKGSRYAPNRGSGGSTLNI | |
| | EDEYRLHETSKEPDVSLGSTWLSDFPQAWA | |
| | ETGGMGLAVRQAPLIIPLKATSTPVSIKQY | |
| | PMSQEARLGIKPHIQRLLDQGILVPCQSPW | |
| | NTPLLPVKKPGTNDYRPVQDLREVNKRVED | |
| | IHPTVPNPYNLLSGLPPSHQWYTVLDLKDA | |
| | FFCLRLHPTSQPLFAFEWRDPEMGISGQLT | |
| | WTRLPQGFKNSPTLFDEALHRDLADFRIQH | |
| | PDLILLQYVDDLLLAATSELDCQQGTRALL | |
| | QTLGNLGYRASAKKAQICQKQVKYLGYLLK | |
| | EGQRWLTEARKETVMGQPTPKTPRQLREFL | |
| | GTAGFCRLWIPGFAEMAAPLYPLTKTGTLF | |
| | NWGPDQQKAYQEIKQALLTAPALGLPDLTK | |
| | PFELFVDEKQGYAKGVLTQKLGPWRRPVAY | |
| | LSKKLDPVAAGWPPCLRMVAAIAVLTKDAG | |
| | KLTMGQPLVILAPHAVEALVKQPPDRWLSN | |
| | ARMTHYQALLLDTDRVQFGPVVALNPATLL | |
| | PLPEEGLQHNCLDILAEAHGTRPDLTDQPL | |
| | PDADHTWYTNGSSLLQEGQRKAGAAVTTET | |
| | EVIWAKALPAGTSAQRAQLIALTQALKMAE | |
| | GKKLNVYTNSRYAFATAHIHGEIYRRRGLL | |
| | TSEGKEIKNKDEILALLKALFLPKRLSIIH | |
| | CPGHQKGHSAEARGNRMANQAARKAAITET | |
| | PDTSTLL | |
| Bos6C23 | MGSSHHHHHHHHGSGGSYPNGTSAALRETG | 92 |
| | VIEKLLTSYGFIQCSERQARLFFHCSQYNG | |
| | NLQDLKVGDDVEFEVSSDRRTGKPIAVKLV | |
| | KIGSGGSTLNIEDEYRLHETSKEPDVSLGS | |
| | TWLSDFPQAWAETGGMGLAVRQAPLIIPLK | |
| | ATSTPVSIKQYPMSQEARLGIKPHIQRLLD | |
| | QGILVPCQSPWNTPLLPVKKPGTNDYRPVQ | |
| | DLREVNKRVEDIHPTVPNPYNLLSGLPPSH | |
| | QWYTVLDLKDAFFCLRLHPTSQPLFAFEWR | |
| | DPEMGISGQLTWTRLPQGFKNSPTLFDEAL | |
| | HRDLADFRIQHPDLILLQYVDDLLLAATSE | |
| | LDCQQGTRALLQTLGNLGYRASAKKAQICQ | |
| | KQVKYLGYLLKEGQRWLTEARKETVMGQPT | |
| | PKTPRQLREFLGTAGFCRLWIPGFAEMAAP | |
| | LYPLTKTGTLFNWGPDQQKAYQEIKQALLT | |
| | APALGLPDLTKPFELFVDEKQGYAKGVLTQ | |
| | KLGPWRRPVAYLSKKLDPVAAGWPPCLRMV | |
| | AAIAVLTKDAGKLTMGQPLVILAPHAVEAL | |
| | VKQPPDRWLSNARMTHYQALLLDTDRVQFG | |
| | PVVALNPATLLPLPEEGLQHNCLDILAEAH | |
| | GTRPDLTDQPLPDADHTWYTNGSSLLQEGQ | |
| | RKAGAAVTTETEVIWAKALPAGTSAQRAQL | |
| | IALTQALKMAEGKKLNVYTNSRYAFATAHI | |
| | HGEIYRRRGLLTSEGKEIKNKDEILALLKA | |
| | LFLPKRLSIIHCPGHQKGHSAEARGNRMAN | |
| | QAARKAAITETPDTSTLL | |

### Example 8

This example illustrates that the abilities of RNA synthesis of Bst, CA2, and CST, all of which are DNA polymerase, have improved with the fusion of Csp.

As shown in FIG. 14, it is illustrated that ttCsp1 can be fused to Bst, CA2, and CST DNA polymerases, which also have reverse transcriptase activity, to increase polymerases processivity with respect to RNA substrates (0.3 kb, 0.5 kb, and 1 kb). These DNA polymerases by themselves can barely reverse transcribe a 0.3 kb RNA substrate at 50° C. for 60′. With ttCsp1 fusion, cBst, cCA2 and cCST can easily reverse transcribe to 1 kb under the same condition.

TABLE 8
Sequences of Bst, CA2, CST and the ttCsp1
Fusion Thereof

| | | SEQ ID |
|---|---|---|
| Name | Sequence | NO: |
| Bst DNA | MGSSHHHHHHHHGSGGSAFTLADRVTEEML | 93 |
| polymerase | ADKAALVVEVVEENYHDAPIVGIAVVNEHG | |
| | RFFLRPETALADPQFVAWLGDETKKKSMFD | |
| | SKRAAVALKWKGIELCGVSFDLLLAAYLLD | |
| | PAQGVDDVAAAAKMKQYEAVRPDEAVYGKG | |
| | AKRAVPDEPVLAEHLVRKAAAIWALERPFL | |
| | DELRRNEQDRLLVELEQPLSSILAEMEFAG | |
| | VKVDTKRLEQMGEELAEQLRTVEQRIYELA | |
| | GQEFNINSPKQLGVILFEKLQLPVLKKTKT | |
| | GYSTSADVLEKLAPYHEIVENILHYRQLGK | |
| | LQSTYIEGLLKVVRPDTKKVHTIFNQALTQ | |
| | TGRLSSTEPNLQNIPIRLEEGRKIRQAFVP | |
| | SESDWLIFAADYSQIELRVLAHIAEDDNLM | |
| | EAFRRDLDIHTKTAMDIFQVSEDEVTPNMR | |
| | RQAKAVNFGIVYGISDYGLAQNLNISRKEA | |
| | AEFIERYFESFPGVKRYMENIVQEAKQKGY | |
| | VTTLLHRRRYLPDITSRNFNVRSFAERMAM | |
| | NTPIQGSAADIIKKAMIDLNARLKEERLQA | |
| | RLLLQVHDELILEAPKEEMERLCRLVPEVM | |
| | EQAVTLRVPLKVDYHYGSTWYDAK | |
| cBst DNA | MGSSHHHHHHHHGSGGSQKGRVKWFNAEKG | 94 |
| polymerase | YGFIEREGDTDVFVHYTAINAKGFRTLNEG | |
| | DIVTFDVEPGRNGKGPQAVNVTVVEPARRG | |
| | SGGSAFTLADRVTEEMLADKAALVVEVVEE | |
| | NYHDAPIVGIAVVNEHGRFFLRPETALADP | |
| | QFVAWLGDETKKKSMFDSKRAAVALKWKGI | |
| | ELCGVSFDLLLAAYLLDPAQGVDDVAAAAK | |
| | MKQYEAVRPDEAVYGKGAKRAVPDEPVLAE | |
| | HLVRKAAAIWALERPFLDELRRNEQDRLLV | |
| | ELEQPLSSILAEMEFAGVKVDTKRLEQMGE | |
| | ELAEQLRTVEQRIYELAGQEFNINSPKQLG | |
| | VILFEKLQLPVLKKTKTGYSTSADVLEKLA | |
| | PYHEIVENILHYRQLGKLQSTYIEGLLKVV | |
| | RPDTKKVHTIFNQALTQTGRLSSTEPNLQN | |
| | IPIRLEEGRKIRQAFVPSESDWLIFAADYS | |
| | QIELRVLAHIAEDDNLMEAFRRDLDIHTKT | |
| | AMDIFQVSEDEVTPNMRRQAKAVNFGIVYG | |
| | ISDYGLAQNLNISRKEAAEFIERYFESFPG | |
| | VKRYMENIVQEAKQKGYVTTLLHRRRYLPD | |
| | ITSRNFNVRSFAERMAMNTPIQGSAADIIK | |
| | KAMIDLNARLKEERLQARLLLQVHDELILE | |
| | APKEEMERLCRLVPEVMEQAVTLRVPLKVD | |
| | YHYGSTWYDAK | |
| CA2 DNA | MGSSHHHHHHHHGSGGSEGVDVRCPDRPEE | 95 |
| polymerase | VEEALSRLEAAQSVVVEVTGDNPHDGEVRG | |
| | VAWWDGHTAYFIPFERLVQSDMRPLADWLA | |
| | DARRPKRTHDSHRAEVALFWHGLAFRGTSF | |
| | CTHIAAYLLDPTESRHTLADLSRRYGLPPV | |
| | PEAEDVYGKGAKFKVPDRDTLARYVGRKAA | |
| | LVARLVPLLEADLAACGMRSLFYDLELPLS | |
| | SELAVMETVGVRVDAAALAAYGEELREAAA | |
| | KVEREIYELAGTTFNIGSTKQLGEILFDKL | |
| | GLPVVKKTKTGYSTDADVLEELAPYHPIVE | |
| | KILHYRQLTKLQSTYIEGLLKEIRPQTGKI | |
| | HTYYQQTIAATGRLSSQFPNLQNIPIRLEE | |
| | GRKIRKAFVPSEPGWLMLAADYSQIELRVL | |
| | AHVSGDERLKEAFRTGMDIHTKTAMDVFGV | |
| | SEDRVDARMRRQAKAVNFGIIYGISDFGLA | |
| | QNLNISRKEAAEFIRQYFAVFSGVKAYRER | |
| | IVEQARRDGYVTTLLGRRRYLPDINASNYN | |
| | LRSFAERTAMNTPIQGTAADIIKTAMVRLT | |
| | RRMRDVGLKSRMLLQVHDELVFEVPPDELD | |
| | AMRELVTDVMESAVPLDVPLKVDVSWGADW | |
| | YAAK | |
| cCA2 DNA | MGSSHHHHHHHHGSGGSQKGRVKWFNAEKG | 96 |
| polymerase | YGFIEREGDTDVFVHYTAINAKGFRTLNEG | |
| | DIVTFDVEPGRNGKGPQAVNVTVVEPARRG | |
| | SGGSEGVDVRCPDRPEEVEEALSRLEAAQS | |
| | VVVEVTGDNPHDGEVRGVAWWDGHTAYFIP | |
| | FERLVQSDMRPLADWLADARRPKRTHDSHR | |
| | AEVALFWHGLAFRGTSFCTHIAAYLLDPTE | |
| | SRHTLADLSRRYGLPPVPEAEDVYGKGAKF | |
| | KVPDRDTLARYVGRKAALVARLVPLLEADL | |
| | AACGMRSLFYDLELPLSSELAVMETVGVRV | |
| | DAAALAAYGEELREAAAKVEREIYELAGTT | |
| | FNIGSTKQLGEILFDKLGLPVVKKTKTGYS | |
| | TDADVLEELAPYHPIVEKILHYRQLTKLQS | |
| | TYIEGLLKEIRPQTGKIHTYYQQTIAATGR | |
| | LSSQFPNLQNIPIRLEEGRKIRKAFVPSEP | |
| | GWLMLAADYSQIELRVLAHVSGDERLKEAF | |
| | RTGMDIHTKTAMDVFGVSEDRVDARMRRQA | |
| | KAVNFGIIYGISDFGLAQNLNISRKEAAEF | |
| | IRQYFAVFSGVKAYRERIVEQARRDGYVTT | |
| | LLGRRRYLPDINASNYNLRSFAERTAMNTP | |
| | IQGTAADIIKTAMVRLTRRMRDVGLKSRML | |
| | LQVHDELVFEVPPDELDAMRELVTDVMESA | |
| | VPLDVPLKVDVSWGADWYAAK | |
| Cst DNA | MGSSHHHHHHHHGSGGSELKITHISAAEDL | 97 |
| polymerase | KKWIAYLLNQKNISVLQLIDREDSYSSRLS | |
| | GLALCTGDEVFYIETGTALPENLIATELKE | |
| | LWQNENIHKIGHNIKEFITWLLKHDVELNG | |
| | LYFDTMIAEYLIDSIRNGYPIASLSHKYLN | |
| | RSVPSLDELLGKGKGAKKYSEIPPERLKDY | |
| | SAYNVKAIFDIWPMQKKVLQENRQEELFND | |
| | IELPLITVLASMEYHGFKVDAAKLHEYGEV | |
| | LLSRIKDLEKVIYMLAGEEFNINSTKQLGT | |
| | ILFEKLKLPVVKSTKTGYSTDVEVLEELYY | |
| | KHDIIPCIIEYRQLTKLYTTYAEGLEKVIN | |
| | PVTGKIHSSFNQTVTATGRISSTEPNLQNI | |
| | PVRHEMGREIRKAFIPSSENAVFVDADYSQ | |
| | IELRVLAHITGDEALINAFVKGEDIHTATA | |
| | SLVFDVAPEDVTPELRRKAKAVNFGIVYGI | |
| | SDYGLARDLGITRKEAKRYIDDYFAKYPKV | |
| | KTYVDEIVRVGQEQGYVETLFHRRRYLPEL | |
| | ASKNFHQRSFGKRVAMNTPIQGTAADIIKI | |
| | AMVKVYKALKESGLKSRLILQVHDELVIET | |
| | FEDELETVKELVKKCMEEAVELSVPLVVDV | |
| | SIGKNWYEAS | |
| cCst DNA | MGSSHHHHHHHHGSGGSQKGRVKWFNAEKG | 98 |
| polymerase | YGFIEREGDTDVFVHYTAINAKGFRTLNEG | |
| | DIVTFDVEPGRNGKGPQAVNVTVVEPARRG | |
| | SGGSELKITHISAAEDLKKWIAYLLNQKNI | |
| | SVLQLIDREDSYSSRLSGLALCTGDEVFYI | |
| | ETGTALPENLIATELKELWQNENIHKIGHN | |
| | IKEFITWLLKHDVELNGLYFDTMIAEYLID | |
| | SIRNGYPIASLSHKYLNRSVPSLDELLGKG | |
| | KGAKKYSEIPPERLKDYSAYNVKAIFDIWP | |
| | MQKKVLQENRQEELFNDIELPLITVLASME | |
| | YHGFKVDAAKLHEYGEVLLSRIKDLEKVIY | |
| | MLAGEEFNINSTKQLGTILFEKLKLPVVKS | |
| | TKTGYSTDVEVLEELYYKHDIIPCIIEYRQ | |
| | LTKLYTTYAEGLEKVINPVTGKIHSSFNQT | |
| | VTATGRISSTEPNLQNIPVRHEMGREIRKA | |
| | FIPSSENAVFVDADYSQIELRVLAHITGDE | |
| | ALINAFVKGEDIHTATASLVFDVAPEDVTP | |
| | ELRRKAKAVNFGIVYGISDYGLARDLGITR | |
| | KEAKRYIDDYFAKYPKVKTYVDEIVRVGQE | |
| | QGYVETLFHRRRYLPELASKNFHQRSFGKR | |
| | VAMNTPIQGTAADIIKIAMVKVYKALKESG | |
| | LKSRLILQVHDELVIETFEDELETVKELVK | |
| | KCMEEAVELSVPLVVDVSIGKNWYEAS | |

1. A fusion protein comprising:
(a) a reverse transcriptase (RT); and
(b) a cold shock protein (Csp) operably linked to the RT.

2. The fusion protein of claim 1, wherein the Csp is linked to N-terminus of the RT.

3. The fusion protein of claim 1, wherein the Csp is linked to C-terminus of the RT.

4. The fusion protein of claim 1, wherein the RT is selected from the group consisting of MMLV RT, AMV RT, HIV RT, WDSV RT, RSV RT, ASLV RT, REV-T RT, MAV RT and RAV RT.

5. The fusion protein of claim 1, wherein the RT is selected from the group consisting of Tth DNA polymerase, Tfl DNA polymerase, Tfi DNA polymerase, Tma DNA polymerase, Tne DNA polymerase, Z05 DNA polymerase, JDF-3 DNA polymerase, Bst DNA polymerase, Bca DNA polymerase, CA2 DNA polymerase and CST DNA polymerase.

6. The fusion protein of claim 1, wherein the RT has an amino acid sequence selected from SEQ ID NOs: 40-48 or a sequence having at least 80% identity thereto with the reverse transcriptase activity.

7. The fusion protein of claim 1, wherein the Csp comprises a cold shock domain (CSD) which comprises at least one motif having a formula selected from the group consisting of: (1) G-X₁-X₂-K-X₃-F-X₄ (SEQ ID NO: 1), (2) X₅-G-X₆-G-X₇-I (SEQ ID NO: 2), (3) X₈-X₉-X₁₀-X₁₁-X₁₂ (SEQ ID NO: 3), (4) X₁₃-X₁₄-X₁₅-X₁₆-X₁₇-X₁₈-X₁₉-X₂₀-X₂₁-X₂₂-X₂₃-X₂₄-X₂₅ (SEQ ID NO: 4), or (5) X₂₆-G-X₂₇-X₂₈-A-X₂₉-X₃₀-X₃₁ (SEQ ID NO: 5), wherein:
X₁ is T, I, V, Q, N, L, K or R;
X₂ is V, I, L, A or G;
X₃ is F, T, Y, H, M or W;
X₄ is N, T, S or D;
X₅ is K, R, S, D, E, N, Q, T, H or Y;
X₆ is F, Y, W, L, V, A, I, M, H, R or K;
X₇ is F, Y, W, L, V, A, I, M or H;
X₈ is V, A, I, L, M, F or W;
X₉ is F, Y, W, H, Q, L, V, I, M or A;
X₁₀ is A, I, L, M, F, V or W;
X₁₁ is H, Y, F, L, M, V, I, W, Q or A;
X₁₂ is Y, W, L, F, M, V, I, Q, H or A;
X₁₃ is G, D, N or E;
X₁₄ is F, I, L, A or Y;
X₁₅ is K, P, Q, E or R;
X₁₆ is T, A, E, V or S;
X₁₇ is L, P or I;
X₁₈ is E, D, T, I, A, F, N or K;
X₁₉ is E, Q, T, P, A or D;
X₂₀ is G or N;
X₂₁ is Q, M, T, L, E or D;
X₂₂ is K, R, N, E, S, Q, T, L, V, A or I;
X₂₃ is V or I;
X₂₄ is E, S, T or Q;
X₂₅ is Y or F;
X₂₆ is K or R;
X₂₇ is P, L, N, Y or A;
X₂₈ is Q, K, S, T, A or H;
X₂₉ is A, V, T, S, G, R or E;
X₃₀ is N, E, K, V, C, R, H, G, D or S;
X₃₁ is V, L or I.

8. The fusion protein of claim 1, wherein the Csp is selected from the group consisting of ahCsp, abCsp, adCsp, atCsp, bbCspD, bsCspC, bsCspD, bsCpsB, bpCspA, ecCspA, ecCspB, ecCspC, ecCspD, ecCspE, ecCspF, ecCspG, ecCspH, ecCspI, mtCspA, msCsp, svCspA, tgCsp, tgemCsp, txCsp, tCsp, tglyCsp, tsCsp, tbCsp, vcCspD, YBX1, YBX2 and CSDE1a.

9. The fusion protein of claim 1, wherein the Csp has an amino acid sequence selected from SEQ ID NOs: 7-39 or a sequence having at least 30% identity thereto.

10. The fusion protein of claim 1, wherein the Csp is linked to the RT via a linker.

11. The fusion protein of claim 10, wherein the linker has an amino acid sequence selected from G, PG, GSG, or any one of SEQ ID NOs: 49-59.

12. The fusion protein of claim 1, which has an amino acid sequence selected from SEQ ID NOs: 78-83, 86-92, 94, 96, 98 or a sequence having at least 80% identity thereto; optionally, the fusion protein has improved activity of reverse transcription processivity.

13. The fusion protein of claim 1, wherein the fusion protein has improved activity of reverse transcription or processivity.

14. A polynucleotide encoding the fusion protein of claim 1.

15. A vector comprising the polynucleotide of claim 14.

16. A recombinant host cell suitable for producing a protein, comprising the polynucleotide of claim 14.

17. A kit for reverse transcription reaction comprising the fusion protein of claim 1 and a reaction buffer solution.

18. The kit of claim 17, further comprising a primer.

19. A method of synthesizing a DNA, comprising:
incubating the fusion protein of claim 1 with an RNA template and a primer under a condition suitable for the fusion protein to perform reverse transcription reaction, thereby synthesizing a DNA strand complementary to the RNA template.

20. The method of claim 19, wherein the primer is an oligo (dT) primer, a random sequence primer, or a combination thereof.


SEQUENCE LISTING (98 sequences)

SEQ ID NO: 1-5 - skipped (5 entries)

SEQ ID NO: 6 - protein, 39 aa, synthetic construct
  VARIANT 3: note = V, L, A, G or I
  VARIANT 5: note = F, T, Y, H, M or W
  VARIANT 2: note = T, I, V, Q, N, L, K or R
  VARIANT 7: note = N, T, S or D
  VARIANT 8: note = K, R, S, D, E, N, Q, T, H or Y
  VARIANT 10: note = F, Y, W, L, V, A, I, M, H, R or K
  VARIANT 12: note = F, Y, W, L, V, A, I, M or H
  VARIANT 14: note = V, A, I, L, M, F or W;
  VARIANT 15: note = F, Y, W, H, Q, L, V, I, M or A;
  VARIANT 16: note = A, I, L, M, F, V or W
  VARIANT 17: note = H, Y, F, L, M, V, I, W, Q or A
  VARIANT 18: note = Y, W, L, F, M, V, I, Q, H or A
  VARIANT 19: note = G, D, N or E
  VARIANT 20: note = F, I, L, A or Y;
  VARIANT 21: note = K, P, Q, E or R
  VARIANT 22: note = T, A, E, V or S
  VARIANT 23: note = L, P or I
  VARIANT 24: note = E, D, T, I, A, F, N or K
  VARIANT 25: note = E, Q, T, P, A or D
  VARIANT 26: note = G or N
  VARIANT 27: note = Q, M, T, L, E or D
  VARIANT 28: note = K, R, N, E, S, Q, T, L, V, A or I
  VARIANT 29: note = V or I
  VARIANT 30: note = E, S, T or Q
  VARIANT 31: note = Y or F
  VARIANT 32: note = K or R
  VARIANT 34: note = P, L, N, Y or A
  VARIANT 35: note = Q, K, S, T, A or H
  VARIANT 37: note = A, V, T, S, G, R or E
  VARIANT 38: note = N, E, K, V, C, R, H, G, D or S; and
  VARIANT 39: note = V, L or I
GXXKXFXXGXGXIXXXXXXXXXXXXXXXXXXXGXXAXXX

SEQ ID NO: 7 - protein, 66 aa, Actinomadura harenae
MAQGTVKWFNADKGYGFIAVDGGADVFVHYSVIQMDGYRSLEQGQRVEFEITQSDRGPQAESVRLL

SEQ ID NO: 8 - protein, 67 aa, unidentified
  source 1..67: note = organism - Alicyclobacillaceae bacterium
MQGRVKWFNPDKGYGFISKDDGEDVFVHYSAIQTQGYRTLEEGQLVEFDIVQGARGPQAANVVPVGP

SEQ ID NO: 9 - protein, 67 aa, Alicyclobacillus dauci
MQQGTVKWFNGDKGFGFISVEGGEDVFVHFSAIQSNGFRSLDEGQRVEFDIVEGPKGPQAANVVVIR

SEQ ID NO: 10 - protein, 67 aa, Alicyclobacillus tolerans
MTQGTVKWFNGDKGFGFISVEGGNDVFVHFSAIQSDGFRTLEEGQVVEFEIVEGQRGPQAANVVVIR

SEQ ID NO: 11 - protein, 66 aa, Bacillus subtilis
MEQGTVKWFNAEKGFGFIERENGDDVFVHFSAIQSDGFKSLDEGQKVSFDVEQGARGAQAANVQKA

SEQ ID NO: 12 - protein, 66 aa, Bacillus subtilis
MQNGKVKWFNNEKGFGFIEVEGGDDVFVHFTAIEGDGYKSLEEGQEVSFEIVEGNRGPQASNVVKL

SEQ ID NO: 13 - protein, 67 aa, Bacillus subtilis
MLEGKVKWFNSEKGFGFIEVEGQDDVFVHFSAIQGEGFKTLEEGQAVSFEIVEGNRGPQAANVTKEA

SEQ ID NO: 14 - protein, 67 aa, Bordetella pertussis
METGVVKWFNAEKGYGFITPEAGGKDLFAHFSEIQANGFKSLEENQRVSFVTAMGPKGPQATKIQIL

SEQ ID NO: 15 - protein, 70 aa, Escherichia coli
MSGKMTGIVKWFNADKGFGFITPDDGSKDVFVHFSAIQNDGYKSLDEGQKVSFTIESGAKGPAAGNVTSL

SEQ ID NO: 16 - protein, 71 aa, Escherichia coli
MSNKMTGLVKWFNADKGFGFISPVDGSKDVFVHFSAIQNDNYRTLFEGQKVTFSIESGAKGPAAANVIITD

SEQ ID NO: 17 - protein, 69 aa, Escherichia coli
MAKIKGQVKWFNESKGFGFITPADGSKDVFVHFSAIQGNGFKTLAEGQNVEFEIQDGQKGPAAVNVTAI

SEQ ID NO: 18 - protein, 74 aa, Escherichia coli
MEKGTVKWFNNAKGFGFICPEGGGEDIFAHYSTIQMDGYRTLKAGQSVQFDVHQGPKGNHASVIVPVEVEAAVA

SEQ ID NO: 19 - protein, 69 aa, Escherichia coli
MSKIKGNVKWFNESKGFGFITPEDGSKDVFVHFSAIQTNGFKTLAEGQRVEFEITNGAKGPSAANVIAL

SEQ ID NO: 20 - protein, 70 aa, Escherichia coli
MSRKMTGIVKTFDGKSGKGLITPSDGRIDVQLHVSALNLRDAEEITTGLRVEFCRINGLRGPSAANVYLS

SEQ ID NO: 21 - protein, 70 aa, Escherichia coli
MSNKMTGLVKWFNADKGFGFITPDDGSKDVFVHFTAIQSNEFRTLNENQKVEFSIEQGQRGPAAANVVTL

SEQ ID NO: 22 - protein, 70 aa, Escherichia coli
MSRKMTGIVKTFDRKSGKGFIIPSDGRKEVQVHISAFTPRDAEVLIPGLRVEFCRVNGLRGPTAANVYLS

SEQ ID NO: 23 - protein, 70 aa, Escherichia coli
MSNKMTGLVKWFNPEKGFGFITPKDGSKDVFVHFSAIQSNDFKTLTENQEVEFGIENGPKGPAAVHVVAL

SEQ ID NO: 24 - protein, 67 aa, Mycobacterium tuberculosis
MPQGTVKWFNAEKGFGFIAPEDGSADVFVHYTEIQGTGFRTLEENQKVEFEIGHSPKGPQATGVRSL

SEQ ID NO: 25 - protein, 67 aa, Mycolicibacterium smegmatis
MPQGTVKWFNAEKGFGFIAPEDGSADVFVHYTEIQGSGFRTLEENQKVEFEVGQSPKGPQATGVRTI

SEQ ID NO: 26 - protein, 70 aa, Shewanella violacea
MSDSNTGTVKWFNEDKGFGFLTQDNGGADVFVHFRAIASEGFKTLDEGQKVTFEVEQGPKGLQASNVIAL

SEQ ID NO: 27 - protein, 97 aa, Tepidamorphus gemmatus
TEPIDAMORPHUSGEMMATUSTGCSPMRQTGTVKFFNQSKGYGFISPDGGGSDVFVHVSDVQRSGIPALDQGMRISYETQPDKRGKGPKAVELQVAG

SEQ ID NO: 28 - protein, 70 aa, Tepidamorphus gemmatus
MRQNGTIKFFNHSRGFGFITPDSGSKDVFVHITALERSGLQAPDEGTKVSFEIEEDRRGRGPQAVNIQLA

SEQ ID NO: 29 - protein, 67 aa, Tepidimicrobium xylanilyticum
MVNGTVKWFNSEKGFGFIKTEEGNDVFVHYSQINKQGFKTLEEGETVSFRIVQGQKGPQAEDVTPIK

SEQ ID NO: 30 - protein, 66 aa, Terrisporobacter sp.
MTNGTVKWFNNDKGFGFISVEGGDDVFAHFSAIKSDGYKSLEEGQKVSFDIVQGARGPQAENITIL

SEQ ID NO: 31 - protein, 66 aa, Terrisporobacter glycolicus
MSNGIVKWFNSEKGFGFITVEGGEDVFAHFSAIQTDGYKTLEEGQKVSFNIVKGARGPQAENITIL

SEQ ID NO: 32 - protein, 66 aa, Terrisporobacter sp.
MNNGIVKWFNNEKGFGFISVEGGDDVFAHFTAIQGEGFKSLEEGQKVSFDIVEGAKGLQAANITIL

SEQ ID NO: 33 - protein, 66 aa, Thermotoga sp.
MKGTVKWFDPKKGYGFITKEEGGDVFVHWSALEMDGFKTLKDGQEVEFEIQEGPKGPQAAHVKVLS

SEQ ID NO: 34 - protein, 68 aa, Thermus thermophilus
MNKGIVKWFNAEKGYGFIQQEEGPDVFVHFSAIEAEGFRTLNEGERVEFEVEPGRNGKGPQARRVRRL

SEQ ID NO: 35 - protein, 73 aa, Thermus thermophilus
MQKGRVKWFNAEKGYGFIEREGDTDVFVHYTAINAKGFRTLNEGDIVTFDVEPGRNGKGPQAVNVTVVEPARR

SEQ ID NO: 36 - protein, 76 aa, Vibrio cholerae
MYSMATGTVKWFNNAKGFGFICPEGEDGDIFAHYSTIQMDGYRTLKAGQQVSYQVEQGPKGYHASCVVPIEGQSAK

SEQ ID NO: 37 - protein, 78 aa, Homo sapiens
DKKVIATKVLGTVKWFNVRNGYGFINRNDTKEDVFVHQTAIKKNNPRKYLRSVGDGETVEFDVVEGEKGAEAANVTGP

SEQ ID NO: 38 - protein, 93 aa, Homo sapiens
ADKPVLATKVLGTVKWFNVRNGYGFINRNDTKEDVFVHQTAIKRNNPRKFLRSVGDGETVEFDVVEGEKGAEATNVTGPGGVPVKGSRYAPNR

SEQ ID NO: 39 - protein, 75 aa, Homo sapiens
YPNGTSAALRETGVIEKLLTSYGFIQCSERQARLFFHCSQYNGNLQDLKVGDDVEFEVSSDRRTGKPIAVKLVKI

SEQ ID NO: 40 - protein, 671 aa, synthetic construct
LNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLI

SEQ ID NO: 41 - protein, 560 aa, synthetic construct
PISPIETVPVKLKPGMDGPKVKQWPLTEEKIKALVEICTEMEKEGKISKIGPENPYNTPVFAIKKKDGTKWRKLVDFRELNKKTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWVPEWEFVNTPPLVKLWYQLEKEPIVGAETFYVDGAASRETKLGKAGYVTNKGRQKVVTLTDTTNQKTELQAIHLALQDSGLEVNIVTNSQYALGIIQAQPDQSESELVNQIIEQLIKKEKVYLAWVPAHKGIGGNEQVDKLVSAGIRKIL

SEQ ID NO: 42 - protein, 858 aa, synthetic construct
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTDASSSTHKGVVVWREGPRWEIKEIADLGASVQQLEARAVAMALLLWPTTPTNVVTDSAFVAKMLLKMGQEGVPSTAAAFILEDALSQRSAMAAVLHVRSHSEVPGFFTEGNDVADSQATFQAYPLREAKDLHTALHIGPRALSKACNISMQQAREVVQTCPHCNSAPALEAGVNPRGLGPLQIWQTDFTLEPRMAPRSWLAVTVDTASSAIVVTQHGRVTSVAAQHHWATAIAVLGRPKAIKTDNGSCFTSKSTREWLARWGIAHTTGIPGNSQGQAMVERANRLLKDKIRVLAEGDGFMKRIPTSKQGELLAKAMYALNHFERGENTKTPIQKHWRPTVLTEGPPVKIRIETGEWEKGWNVLVWGRGYAAVKNRDTDKVIWVPSRKVKPDIAQKDEVTKKDEASPLFA

SEQ ID NO: 43 - protein, 834 aa, synthetic construct
MEAMLPLFEPKGRVLLVDGHHLAYRTFFALKGLTTSRGEPVQAVYGFAKSLLKALKEDGYKAVFVVFDAKAPSFRHEAYEAYKAGRAPTPEDFPRQLALIKELVDLLGFTRLEVPGYEADDVLATLAKKAEKEGYEVRILTADRDLYQLVSDRVAVLHPEGHLITPEWLWEKYGLRPEQWVDFRALVGDPSDNLPGVKGIGEKTALKLLKEWGSLENLLKNLDRVKPENVREKIKAHLEDLRLSLELSRVRTDLPLEVDLAQGREPDREGLRAFLERLEFGSLLHEFGLLEAPAPLEEAPWPPPEGAFVGFVLSRPEPMWAELKALAACRDGRVHRAADPLAGLKDLKEVRGLLAKDLAVLASREGLDLVPGDDPMLLAYLLDPSNTTPEGVARRYGGEWTEDAAHRALLSERLHRNLLKRLEGEEKLLWLYHEVEKPLSRVLAHMEATGVRRDVAYLQALSLELAEEIRRLEEEVFRLAGHPFNLNSRDQLERVLFDELRLPALGKTQKTGKRSTSAAVLEALREAHPIVEKILQHRELTKLKNTYVDPLPSLVHPRTGRLHTRFNQTATATGRLSSSDPNLQNIPVRTPLGQRIRRAFVAEAGWALVALDYSQIELRVLAHLSGDENLIRVFQEGKDIHTQTASWMFGVPPEAVDPLMRRAAKTVNFGVLYGMSAHRLSQELAIPYEEAVAFIERYFQSFPKVRAWIEKTLEEGRKRGYVETLFGRRRYVPDLNARVKSVREAAERMAFNMPVQGTAADLMKLAMVKLFPRLREMGARMLLQVHDELLLEAPQARAEEVAALAKEAMEKAYPLAVPLEVEVGMGEDWLSAKG

SEQ ID NO: 44 - protein, 828 aa, synthetic construct
MAMLPLFEPKGRVLLVDGHHLAYRTFFALKGLTTSRGEPVQAVYGFAKSLLKALKEDGDVVVVVFDAKAPSFRHEAYEAYKAGRAPTPEDFPRQLALIKELVDLLGLVRLEVPGFEADDVLATLAKRAEKEGYEVRILTADRDLYQLLSERIAILHPEGYLITPAWLYEKYGLRPEQWVDYRALAGDPSDNIPGVKGIGEKTAQRLIREWGSLENLFQHLDQVKPSLREKLQAGMEALALSRKLSKVHTDLPLEVDFGRRRTPNLEGLRAFLERLEFGSLLHEFGLLEGPKAAEEAPWPPEGAFLGFSFSRPEPMWAELLALAGAWEGRLFRAQDPLRGLRDLKGVRGILAKDLAVLALREGLDLFPEDDPMLLAYLLDPSNTTPEGVARRYGGEWTEDAGERALLAERLFQTLKERLYGEERLLWLYEEVEKPLSRVLARMEATGVRLDVAYLQALSLEVEAEVRQLEEEVFRLAGHPFNLNSRDQLERVLFDELGLPAIGKTEKTGKRSTSAAVLEALREAHPIVDRILQYRELTKLKNTYIDPLPALVHPKTGRLHRRFNQTATTGRLSSSDPNLQNIPVRTVLGQRIRRAFVAEEGWVLVVLDYSQIERLVLAHLSGTENLIRVFQEGREIHTQTASWMFGVSPEGVDPLMRRAAKTINFGVLYGMSAHRLSGELSIPYEEAVAFIERYFQSYPKVRAWIEGTLEEGRRRGYVETLFGRRRYVPDLNARVKSVREAAERMAFNNPVQGAADLMKLAMVRLFPRLQELGARMLLQVHDELVLEAPKDRAERVAALAKEVMEGVWPLQVPLEVEVGLGEDWLSAKE

SEQ ID NO: 45 - protein, 831 aa, synthetic construct
MTPLFDLEEPPKRVLLVDGHHLAYRTFYALSLTTSRGEPVQMVYGFARSLLKALKEDGQAVVVVFDAKAPSFRHEAYEAYKAGRAPTPEDFPRQLALVKRLVDLLGLVRLEAPGYEADDVLGTLAKKAEREGMEVRILTGDRDFFQLLSEKVSVLLPDGTLVTPKVQEKYGVPPERWVDFRALTGDRSDNIPGVAGIGEKTALRLLAEWGSVENLLKNLDRVKPDSVRRKIEAHLEDLRLSLDLARIRTDLPLEVDFKALRRRTPDLEGLRAFLEELEFGSLLHEFGLLGGEKPREEAPWPPPEGAFVGFLLSRKEPMWAELLALAAAAEGRVHRATSPVEALADLKEARGFLAKDLAVLALREGVALDPTDDPLLVAYLLDPANTNPEGVARRYGGEFTEDAAERALLSERLFQNLFERLSEKLLWLYQEVERPLSRVLAHMEARGVRLDVPLLEALSFELEKEMERLEGEVFRLAGHPFNLNSRDQLERVLFDELGLTPVGRTEKTGRSTAQGALEALRGAHPIVELILQYRELSKLKSTYLDPLPRLVHPRTGRLHTRFNQTATATGRLSSSTPNLQNIPVRTPLGQRIRKAFVAEEGWLLLAADYSQIELRVLAHLSGDENLKRVFREGKDIHTETAAWMFGLDPALVDPKMRRAAKTVNFGVLYGMSAHRLSQELGIDYKEAEAFIERYFQSFPKVRAWIERTLEEGRTRGYVETLFGRRRYVPDLASRVRSVREAAERMAFNMPVQGTAADLMKIAMVKLFPRLKPLGAHLLLQVHDELVLEVPEDRAEEAKALVKEVMENTYPLDVPLEVEVGVGRDWLEAKGD

SEQ ID NO: 46 - protein, 577 aa, synthetic construct
AFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWALERPFLDELRRNEQDRLLVELEQPLSSILAEMEFAGVKVDTKRLEQMGEELAEQLRTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLQVHDELILEAPKEEMERLCRLVPEVMEQAVTLRVPLKVDYHYGSTWYDAK

SEQ ID NO: 47 - protein, 587 aa, synthetic construct
EGVDVRCPDRPEEVEEALSRLEAAQSVVVEVTGDNPHDGEVRGVAWWDGHTAYFIPFERLVQSDMRPLADWLADARRPKRTHDSHRAEVALFWHGLAFRGTSFCTHIAAYLLDPTESRHTLADLSRRYGLPPVPEAEDVYGKGAKFKVPDRDTLARYVGRKAALVARLVPLLEADLAACGMRSLFYDLELPLSSELAVMETVGVRVDAAALAAYGEELREAAAKVEREIYELAGTTFNIGSTKQLGEILFDKLGLPVVKKTKTGYSTDADVLEELAPYHPIVEKILHYRQLTKLQSTYIEGLLKEIRPQTGKIHTYYQQTIAATGRLSSQFPNLQNIPIRLEEGRKIRKAFVPSEPGWLMLAADYSQIELRVLAHVSGDERLKEAFRTGMDIHTKTAMDVFGVSEDRVDARMRRQAKAVNFGIIYGISDFGLAQNLNISRKEAAEFIRQYFAVFSGVKAYRERIVEQARRDGYVTTLLGRRRYLPDINASNYNLRSFAERTAMNTPIQGTAADIIKTAMVRLTRRMRDVGLKSRMLLQVHDELVFEVPPDELDAMRELVTDVMESAVPLDVPLKVDVSWGADWYAAK

SEQ ID NO: 48 - protein, 593 aa, synthetic construct
ELKITHISAAEDLKKWIAYLLNQKNISVLQLIDREDSYSSRLSGLALCTGDEVFYIETGTALPENLIATELKELWQNENIHKIGHNIKEFITWLLKHDVELNGLYFDTMIAEYLIDSIRNGYPIASLSHKYLNRSVPSLDELLGKGKGAKKYSEIPPERLKDYSAYNVKAIFDIWPMQKKVLQENRQEELFNDIELPLITVLASMEYHGFKVDAAKLHEYGEVLLSRIKDLEKVIYMLAGEEFNINSTKQLGTILFEKLKLPVVKSTKTGYSTDVEVLEELYYKHDIIPCIIEYRQLTKLYTTYAEGLEKVINPVTGKIHSSFNQTVTATGRISSTEPNLQNIPVRHEMGREIRKAFIPSSENAVFVDADYSQIELRVLAHITGDEALINAFVKGEDIHTATASLVFDVAPEDVTPELRRKAKAVNFGIVYGISDYGLARDLGITRKEAKRYIDDYFAKYPKVKTYVDEIVRVGQEQGYVETLFHRRRYLPELASKNFHQRSFGKRVAMNTPIQGTAADIIKIAMVKVYKALKESGLKSRLILQVHDELVIETFEDELETVKELVKKCMEEAVELSVPLVVDVSIGKNWYEAS

SEQ ID NO: 49 - protein, 5 aa, synthetic construct
GSGGS

SEQ ID NO: 50 - protein, 5 aa, synthetic construct
GGGGS

SEQ ID NO: 51 - protein, 8 aa, synthetic construct
GSGSGSGS

SEQ ID NO: 52 - protein, 10 aa, synthetic construct
GSGGSGSGGS

SEQ ID NO: 53 - protein, 15 aa, synthetic construct
GSGGSGSGGSGSGGS

SEQ ID NO: 54 - protein, 11 aa, synthetic construct
GQGQGQGQGQG

SEQ ID NO: 55 - protein, 12 aa, synthetic construct
HHHHPGGSVKKR

SEQ ID NO: 56 - protein, 6 aa, synthetic construct
GSIEGR

SEQ ID NO: 57 - protein, 6 aa, synthetic construct
SAPGTP

SEQ ID NO: 58 - protein, 8 aa, synthetic construct
SAPGTPSR

SEQ ID NO: 59 - protein, 14 aa, synthetic construct
EGKSSGSGSESKEF

SEQ ID NO: 60 - DNA, 160 bp, Synthetic construct
  source 1..160: mol_type = other DNA
aagcttgaaattaatacgactcactataggggactagtttaattaagtgatccgacaggttacggggtcctgtccgaaaaaaaaagaaaaaaaaaaaactagcataaccccttggggcctctaaacgggtcttgaggggttttttggcggccgctctaga

SEQ ID NO: 61 - DNA, 51 bp, synthetic construct
  source 1..51: mol_type = other DNA
tgaaattaatacgactcactataggggactccgccacgacgatgaacagac

SEQ ID NO: 62 - DNA, 49 bp, synthetic construct
  source 1..49: mol_type = other DNA
ccgtaacctgtcggatcacttaattaaactgtctgcgcgggcattgcca

SEQ ID NO: 63 - DNA, 48 bp, synthetic construct
  source 1..48: mol_type = other DNA
ccgtaacctgtcggatcacttaattaaactcgcccacgactcgttcgc

SEQ ID NO: 64 - DNA, 52 bp, synthetic construct
  source 1..52: mol_type = other DNA
ccgtaacctgtcggatcacttaattaaactgcgcagcaccgtaattactgtg

SEQ ID NO: 65 - DNA, 49 bp, synthetic construct
  source 1..49: mol_type = other DNA
ccgtaacctgtcggatcacttaattaaactcgctgtgcgtccgcatcct

SEQ ID NO: 66 - DNA, 53 bp, synthetic construct
  source 1..53: mol_type = other DNA
ccgtaacctgtcggatcacttaattaaactctccccttataaacccacagggt

SEQ ID NO: 67 - DNA, 51 bp, synthetic construct
  source 1..51: mol_type = other DNA
ccgtaacctgtcggatcacttaattaaactgcctgattgccggaaaggacc

SEQ ID NO: 68 - DNA, 50 bp, synthetic construct
  source 1..50: mol_type = other DNA
ggtcctttccggcaatcaggcagtttaatagggctgacgtttaaccagac

SEQ ID NO: 69 - DNA, 43 bp, synthetic construct
  source 1..43: mol_type = other DNA
ctgcgtgagtatccgtgagaataagtgatccgacaggttacgg

SEQ ID NO: 70 - DNA, 50 bp, synthetic construct
  source 1..50: mol_type = other DNA
tgaaattaatacgactcactataggggactgcgacactccaccatgaatc

SEQ ID NO: 71 - DNA, 50 bp, synthetic construct
  source 1..50: mol_type = other DNA
ccgtaacctgtcggatcacttaattaaactaggaattgcgcacttggtag

SEQ ID NO: 72 - DNA, 50 bp, synthetic construct
  source 1..50: mol_type = other DNA
ccgtaacctgtcggatcacttaattaaactggtggcctggtgttgttaag

SEQ ID NO: 73 - DNA, 50 bp, synthetic construct
  source 1..50: mol_type = other DNA
ccgtaacctgtcggatcacttaattaaactcgaagatggccaggagtagt

SEQ ID NO: 74 - DNA, 50 bp, synthetic construct
  source 1..50: mol_type = other DNA
ccgtaacctgtcggatcacttaattaaactacgccctcccaaaattcaag

SEQ ID NO: 75 - DNA, 50 bp, synthetic construct
  source 1..50: mol_type = other DNA
ccgtaacctgtcggatcacttaattaaacttcatcctcctctgccacaag

SEQ ID NO: 76 - DNA, 50 bp, synthetic construct
  source 1..50: mol_type = other DNA
ccgtaacctgtcggatcacttaattaaactcgagcaggagtaggcaaaac

SEQ ID NO: 77 - protein, 761 aa, synthetic construct
MGSSHHHHHHHHGSGGSMQKGRVKWFNAEKGYGFIEREGDTDVFVHYTAINAKGFRTLNEGDIVTFDVEPGRNGKGPQAVNVTVVEPARRTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMANQAARKAAITETPDTSTLL

SEQ ID NO: 78 - protein, 762 aa, synthetic construct
MGSSHHHHHHHHGSGGSMQKGRVKWFNAEKGYGFIEREGDTDVFVHYTAINAKGFRTLNEGDIVTFDVEPGRNGKGPQAVNVTVVEPARRGTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMANQAARKAAITETPDTSTLL

SEQ ID NO: 79 - protein, 764 aa, synthetic construct
MGSSHHHHHHHHGSGGSMQKGRVKWFNAEKGYGFIEREGDTDVFVHYTAINAKGFRTLNEGDIVTFDVEPGRNGKGPQAVNVTVVEPARRGSGTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMANQAARKAAITETPDTSTLL

SEQ ID NO: 80 - protein, 766 aa, synthetic construct
MGSSHHHHHHHHGSGGSMQKGRVKWFNAEKGYGFIEREGDTDVFVHYTAINAKGFRTLNEGDIVTFDVEPGRNGKGPQAVNVTVVEPARRGSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMANQAARKAAITETPDTSTLL

SEQ ID NO: 81 - protein, 771 aa, synthetic construct
MGSSHHHHHHHHGSGGSMQKGRVKWFNAEKGYGFIEREGDTDVFVHYTAINAKGFRTLNEGDIVTFDVEPGRNGKGPQAVNVTVVEPARRGSGGSGSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMANQAARKAAITETPDTSTLL

SEQ ID NO: 82 - protein, 771 aa, synthetic construct; identical to SEQ ID NO: 81

SEQ ID NO: 83 - protein, 776 aa, synthetic construct
MGSSHHHHHHHHGSGGSMQKGRVKWFNAEKGYGFIEREGDTDVFVHYTAINAKGFRTLNEGDIVTFDVEPGRNGKGPQAVNVTVVEPARRGSGGSGSGGSGSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMANQAARKAAITETPDTSTLL

SEQ ID NO: 84 - protein, 645 aa, synthetic construct
MGSQKGRVKWFNAEKGYGFIEREGDTDVFVHYTAINAKGFRTLNEGDIVTFDVEPGRNGKGPQAVNVTVVEPARRGSGGSGSGGSPISPIETVPVKLKPGMDGPKVKQWPLTEEKIKALVEICTEMEKEGKISKIGPENPYNTPVFAIKKKDGTKWRKLVDFRELNKKTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLSKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWVPEWEFVNTPPLVKLWYQLEKEPIVGAETFYVNGAASRETKLGKAGYVTNKGRQKVVTLTDTTNQKTQLQAIHLALQDSGLEVNIVTNSQYALGIIQAQPDQSESELVNQIIEQLIKKEKVYLAWVPAHKGIGGNEQVDKLVSAGIRKIL

SEQ ID NO: 85 - protein, 444 aa, synthetic construct
MGPISPIETVPVKLKPGMDGPKVKQWPLTEEKIKALVEICTEMEKEGKISKIGPENPYNTPVFAIKKKDGTKWRKLVDFRELNKKTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLSKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWVPEWEFVNTPPLVKLWYQGSGGSSHHHHHHHH

SEQ ID NO: 86 - protein, 763 aa, synthetic construct
MGSSHHHHHHHHGSGGSMSGKMTGIVKWFNADKGFGFITPDDGSKDVFVHFSAIQNDGYKSLDEGQKVSFTIESGAKGPAAGNVTSLGSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMANQAARKAAITETPDTSTLL

SEQ ID NO: 87 - protein, 767 aa, synthetic construct
MGSSHHHHHHHHGSGGSMEKGTVKWFNNAKGFGFICPEGGGEDIFAHYSTIQMDGYRTLKAGQSVQFDVHQGPKGNHASVIVPVEVEAAVAGSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMANQAARKAAITETPDTSTLL

SEQ ID NO: 88 - protein, 763 aa, synthetic construct
MGSSHHHHHHHHGSGGSMSRKMTGIVKTFDRKSGKGFIIPSDGRKEVQVHISAFTPRDAEVLIPGLRVEFCRVNGLRGPTAANVYLSGSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMANQAARKAAITETPDTSTLL

SEQ ID NO: 89 - protein, 764 aa, synthetic construct
MGSSHHHHHHHHGSGGSMAQGTVKWFNADKGYGFIAVDGGADVFVHYSVIQMDGYRSLEQGQRVEFEITQSDRGPQAESVRLLGSGGSGSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMANQAARKAAITETPDTSTLL

SEQ ID NO: 90 - protein, 771 aa, synthetic construct
MGSSHHHHHHHHGSGGSDKKVIATKVLGTVKWFNVRNGYGFINRNDTKEDVFVHQTAIKKNNPRKYLRSVGDGETVEFDVVEGEKGAEAANVTGPGSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMANQAARKAAITETPDTSTLL

SEQ ID NO: 91 - protein, 786 aa, synthetic construct
MGSSHHHHHHHHGSGGSADKPVLATKVLGTVKWFNVRNGYGFINRNDTKEDVFVHQTAIKRNNPRKFLRSVGDGETVEFDVVEGEKGAEATNVTGPGGVPVKGSRYAPNRGSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMANQAARKAAITETPDTSTLL

SEQ ID NO: 92 - protein, 768 aa, synthetic construct
MGSSHHHHHHHHGSGGSYPNGTSAALRETGVIEKLLTSYGFIQCSERQARLFFHCSQYNGNLQDLKVGDDVEFEVSSDRRTGKPIAVKLVKIGSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMANQAARKAAITETPDTSTLL

SEQ ID NO: 93 - protein, 594 aa, synthetic construct
MGSSHHHHHHHHGSGGSAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWALERPFLDELRRNEQDRLLVELEQPLSSILAEMEFAGVKVDTKRLEQMGEELAEQLRTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLQVHDELILEAPKEEMERLCRLVPEVMEQAVTLRVPLKVDYHYGSTWYDAK

SEQ ID NO: 94 - protein, 671 aa, synthetic construct
MGSSHHHHHHHHGSGGSQKGRVKWFNAEKGYGFIEREGDTDVFVHYTAINAKGFRTLNEGDIVTFDVEPGRNGKGPQAVNVTVVEPARRGSGGSAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWALERPFLDELRRNEQDRLLVELEQPLSSILAEMEFAGVKVDTKRLEQMGEELAEQLRTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLQVHDELILEAPKEEMERLCRLVPEVMEQAVTLRVPLKVDYHYGSTWYDAK

SEQ ID NO: 95 - protein, 604 aa, synthetic construct
MGSSHHHHHHHHGSGGSEGVDVRCPDRPEEVEEALSRLEAAQSVVVEVTGDNPHDGEVRGVAWWDGHTAYFIPFERLVQSDMRPLADWLADARRPKRTHDSHRAEVALFWHGLAFRGTSFCTHIAAYLLDPTESRHTLADLSRRYGLPPVPEAEDVYGKGAKFKVPDRDTLARYVGRKAALVARLVPLLEADLAACGMRSLFYDLELPLSSELAVMETVGVRVDAAALAAYGEELREAAAKVEREIYELAGTTFNIGSTKQLGEILFDKLGLPVVKKTKTGYSTDADVLEELAPYHPIVEKILHYRQLTKLQSTYIEGLLKEIRPQTGKIHTYYQQTIAATGRLSSQFPNLQNIPIRLEEGRKIRKAFVPSEPGWLMLAADYSQIELRVLAHVSGDERLKEAFRTGMDIHTKTAMDVFGVSEDRVDARMRRQAKAVNFGIIYGISDFGLAQNLNISRKEAAEFIRQYFAVFSGVKAYRERIVEQARRDGYVTTLLGRRRYLPDINASNYNLRSFAERTAMNTPIQGTAADIIKTAMVRLTRRMRDVGLKSRMLLQVHDELVFEVPPDELDAMRELVTDVMESAVPLDVPLKVDVSWGADWYAAK

SEQ ID NO: 96 - protein, 681 aa, synthetic construct
MGSSHHHHHHHHGSGGSQKGRVKWFNAEKGYGFIEREGDTDVFVHYTAINAKGFRTLNEGDIVTFDVEPGRNGKGPQAVNVTVVEPARRGSGGSEGVDVRCPDRPEEVEEALSRLEAAQSVVVEVTGDNPHDGEVRGVAWWDGHTAYFIPFERLVQSDMRPLADWLADARRPKRTHDSHRAEVALFWHGLAFRGTSFCTHIAAYLLDPTESRHTLADLSRRYGLPPVPEAEDVYGKGAKFKVPDRDTLARYVGRKAALVARLVPLLEADLAACGMRSLFYDLELPLSSELAVMETVGVRVDAAALAAYGEELREAAAKVEREIYELAGTTFNIGSTKQLGEILFDKLGLPVVKKTKTGYSTDADVLEELAPYHPIVEKILHYRQLTKLQSTYIEGLLKEIRPQTGKIHTYYQQTIAATGRLSSQFPNLQNIPIRLEEGRKIRKAFVPSEPGWLMLAADYSQIELRVLAHVSGDERLKEAFRTGMDIHTKTAMDVFGVSEDRVDARMRRQAKAVNFGIIYGISDFGLAQNLNISRKEAAEFIRQYFAVFSGVKAYRERIVEQARRDGYVTTLLGRRRYLPDINASNYNLRSFAERTAMNTPIQGTAADIIKTAMVRLTRRMRDVGLKSRMLLQVHDELVFEVPPDELDAMRELVTDVMESAVPLDVPLKVDVSWGADWYAAK

SEQ ID NO: 97 - protein, 610 aa, synthetic construct
MGSSHHHHHHHHGSGGSELKITHISAAEDLKKWIAYLLNQKNISVLQLIDREDSYSSRLSGLALCTGDEVFYIETGTALPENLIATELKELWQNENIHKIGHNIKEFITWLLKHDVELNGLYFDTMIAEYLIDSIRNGYPIASLSHKYLNRSVPSLDELLGKGKGAKKYSEIPPERLKDYSAYNVKAIFDIWPMQKKVLQENRQEELFNDIELPLITVLASMEYHGFKVDAAKLHEYGEVLLSRIKDLEKVIYMLAGEEFNINSTKQLGTILFEKLKLPVVKSTKTGYSTDVEVLEELYYKHDIIPCIIEYRQLTKLYTTYAEGLEKVINPVTGKIHSSFNQTVTATGRISSTEPNLQNIPVRHEMGREIRKAFIPSSENAVFVDADYSQIELRVLAHITGDEALINAFVKGEDIHTATASLVFDVAPEDVTPELRRKAKAVNFGIVYGISDYGLARDLGITRKEAKRYIDDYFAKYPKVKTYVDEIVRVGQEQGYVETLFHRRRYLPELASKNFHQRSFGKRVAMNTPIQGTAADIIKIAMVKVYKALKESGLKSRLILQVHDELVIETFEDELETVKELVKKCMEEAVELSVPLVVDVSIGKNWYEAS

SEQ ID NO: 98 - protein, 687 aa, synthetic construct
MGSSHHHHHHHHGSGGSQKGRVKWFNAEKGYGFIEREGDTDVFVHYTAINAKGFRTLNEGDIVTFDVEPGRNGKGPQAVNVTVVEPARRGSGGSELKITHISAAEDLKKWIAYLLNQKNISVLQLIDREDSYSSRLSGLALCTGDEVFYIETGTALPENLIATELKELWQNENIHKIGHNIKEFITWLLKHDVELNGLYFDTMIAEYLIDSIRNGYPIASLSHKYLNRSVPSLDELLGKGKGAKKYSEIPPERLKDYSAYNVKAIFDIWPMQKKVLQENRQEELFNDIELPLITVLASMEYHGFKVDAAKLHEYGEVLLSRIKDLEKVIYMLAGEEFNINSTKQLGTILFEKLKLPVVKSTKTGYSTDVEVLEELYYKHDIIPCIIEYRQLTKLYTTYAEGLEKVINPVTGKIHSSFNQTVTATGRISSTEPNLQNIPVRHEMGREIRKAFIPSSENAVFVDADYSQIELRVLAHITGDEALINAFVKGEDIHTATASLVFDVAPEDVTPELRRKAKAVNFGIVYGISDYGLARDLGITRKEAKRYIDDYFAKYPKVKTYVDEIVRVGQEQGYVETLFHRRRYLPELASKNFHQRSFGKRVAMNTPIQGTAADIIKIAMVKVYKALKESGLKSRLILQVHDELVIETFEDELETVKELVKKCMEEAVELSVPLVVDVSIGKNWYEAS